(19) United States
(12) Patent Application Publication
(10) Pub. No.: US 2021/0324486 A1
(43) Pub. Date: Oct. 21, 2021

(54) Method and Primers for the Sensitive and Specific Detection of SARS-CoV-2

(71) Applicant: Thomas Jefferson University, Philadelphia, PA (US)

(72) Inventor: Isidore Rigoutsos, Astoria, NY (US)

(21) Appl. No.: 17/235,348

(22) Filed: Apr. 20, 2021

Related U.S. Application Data
(60) Provisional application No. 63/012,750, filed on Apr. 20, 2020.

(51) Int. Cl. C12Q 1/70

(52) U.S. Cl. CPC C12Q 1/701

(57) ABSTRACT

In various aspects and embodiments the invention provides a method of detecting SARS-CoV-2 in a sample obtained from a subject, the method comprising contacting the sample with a with a composition comprising a plurality of PCR primers targeting at least one sequence selected from the group consisting of SEQ ID NOS: 1-559, thereby forming a PCR mixture; conducting PCR amplification of the PCR mixture and detection of the at least one sequence selected from the group consisting of SEQ ID NOS: 1-559; wherein an elevated level of the at least one sequence selected from the group consisting of SEQ ID NOS: 1-559 indicates the presence of SARS-CoV-2 in the sample.

### CROSS-REFERENCE TO RELATED APPLICATION

This application claims the benefit of priority under 35 U.S.C. § 119(e) to U.S. Provisional Patent Application Ser. No. 63/012,750 filed Apr. 20, 2020. The entire content of this application is hereby incorporated by reference herein.

### BACKGROUND OF THE INVENTION

SARS-CoV-2 is a newly emerged virus that is presently the cause of enormous morbidity and mortality. There is a need in the art for methods for the sensitive and specific detection of SARS-CoV-2. This disclosure addresses that need.

### SEQUENCE LISTING

The ASCII text file named “205961-7059US1(00207) Sequence Listing” created on Apr. 20, 2021, comprising 145 Kbytes, is hereby incorporated by reference in its entirety.

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplified materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “PCR” or “PCR amplification” refers to the polymerase chain reaction used to amplify nucleic acids. The term includes all variants of the polymerase chain reaction, including but not limited to quantitative PCR.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Methods of Detecting SARS-CoV-2

The invention depends in part on the unexpected discovery of certain sites within the genome of SARS-CoV-2 that are useful binding sites for primers used in the detection of the virus. Accordingly, in one aspect the invention a method of detecting SARS-CoV-2 in a sample obtained from a subject, the method by contacting the sample with a composition comprising a plurality of PCR primers targeting at least one sequence selected from the group consisting of SEQ ID NOS: 1-559, thereby forming a PCR mixture; and conducting PCR amplification of the PCR mixture and detection of the at least one sequence selected from the group consisting of SEQ ID NOS: 1-559; wherein an elevated level of the at least one sequence selected from the group consisting of SEQ ID NOS: 1-559 indicates the presence of SARS-CoV-2 in the sample. In various embodiments, the sample is a blood sample or a saliva sample. In various embodiments, the method is a polymerase chain reaction (PCR) based method. In various embodiments the method further comprises analyzing the biological sample by quantitative PCR.

Below is a map of the SARS-CoV2-Wuhan-Hu-1 genome. The regions in capital boldface are the regions against which one can design good quality (sensitive and specific) q-PCR primers.

SEQ ID NO: 560
attaaaggtTTATACCTTCCCAGGTAAcaaaccaaccaactttcgatctcttgtagatctgttctctaaacgaa
ctttaaaatctgtgtggctGTCACTCGGCTGCATGCTTAGTGCACTCACGCAGtataattaataactaattact
gtcgttgacaggacacgagtaactcgtctatcttctgcAGGCTGCTTACGGTTTCGTccgtgttgcagccgatc
atcagcacatctaggtttcgtccgggtgtgaccgaaaggtaagatggagagccttgtccctggtttcaacgaga
aaacacacgtccaactcagtttgcctgTTTTACAGGTTCGCGACGTGCTcgtacgtggctttggagactccgtg
gaggaGGTCTTATCAGAGGCACGTcaacatcttaaagatggcacttgtggcttagtagaagttgaaaaaggcgt
tttgcctcaacttgaacagccctatgtgttCATCAAACGTTCGGATGCTCGAACTGcacctcatggtcatgtta
tggttgagcTGGTAGCAGAACTCGAAGGCATTCAGTACGGTCGTAGTGGTGAGACActtggtgtccttgtCCCT
CATGTGGGCGAAATACCAGTGGCTTACCGCAAGGTTCTTCTTCGTAAGAACGGTaataaaggagctggtggcca
tagttacggcgccgatctaaagtcatttgacttaggcgacgagcttggcactgatccttatgaagattttcaag
aaaactggaacactaaacatagcagtggtgttacccgtgaactCATGCGTGAGCTTAACGGAGGGGCATACACT
CGCTATGTCGATAAcaacttctgtggccctgaTGGCTACCCTCTTGAGTGcattaaagaccttctagcacgtgc
tggtaaagcttcatgcactttgtccgaacaactggactttattgacactaagaggggtgtatactgctgccgtg
aacatgagcatgaaattgcttggtacacggaacgttctgaaaagagctatgaattgcagacaccttttgaaatt
aaattggcaaagaaatttgacaccttcaatggggaatgtccaaattttgtatttcccttaaattccataatcaa
gactattcaaccaagggttgaaaagaaaaagcttgatggctttATGGGTAGAATTCGATCTgtctatcCAGTTG
CGTCACCAAATGaatgcaaccaaatgtgcctttcaactctcatgaagtgtgatcattgtggtgaaacttcatgg
cagacgggcgattttgttaaagccacttgcgaattttgtggcactgagaatttgactaaagaaggtgccactac
ttgtggttacttaccccaaaatgctgttgttaaaatttattgtccagcatgtcacaattcagaagtaggaccTG
AGCATAGTCTTGCCGAATACCATAAtgaatctggcttgaaaaccATTCTTCGTAAGGGTGGTCgcactattgcc
tttggaggctgtgtgttctcttatgttggttgccataacaagtgtgcctattgggTTCCACGTGCTAGCGCTAa
cataggttgtaaccatacaggtgttgttggagaaggttccgaaggtcttaatgacaaccttcttgaaatactcc
aaaaagagaaagtcaacatcaatattgttggtgactttaaacttaatgaagagatcgccattattttggcatct
ttttctgcttccacaagtgcttttgtggaaactgtgaaaggtttggattataaagcattcaaacaaattgttga
atcctgtggtaattttaaagttacaaaaggaaaagctaaaaaaggtgcctggaatattggtgaacagaaatcaa
tactgagtcctctttatgcatttgcatcagaGGCTGCTCGTGTTGTACGATCAATTTTCTcccgcactcttgaa
actgctcaaaattctgtgcgtgttttacagaaggccgctataacaatactagatggaatttcacagtattcact
gagactcattgatgctatgatgttcacatctgatttggctactaacaatctagttgtaatggcctacattacag
gtggtgttgttcagTTGACTTCGCAGTGGCTAACTAacatctttggcactgtttatgaaAAACTCAAACCCGTC
CTTGattggcttgaagagaagtttaaggaaggtgtagagttTCTTAGAGACGGTTGGGAAAttgttaaatttat
ctcaacctgtgcttgtgaaattgtcggtggacaaattgtcacctgtgcaaaggaaattaaggagagtgttcaga
cattctttaagcttgtaaataaatttttggctttgtgtgctgactctatcattattggtggagctaaacttaaa
gccttgaatttaggtgaaacatttgtcacgcactcaaagggattgtacagaaagtgtgttaaatccagagaaga
aactggcctactcatgcctctaaaagccccaaaagaaattatcttcttagagggagaaacacttcccacagaag
tgttaacagaggaagttgtcttgaaaactggtgatttacaaccattagaacaacctactagtgaagctgttgaa
gctccattggttggtacaccagtttgtattaacgggcttatgttgctcgaaatcaaagacacagaaaagtactg
tgcccttgcacctaatatgatggtaacaaacaataccttcacactcaaaggcggtgcaccaacaaaggttactt
ttggtgatgacactgtgatagaagtgcaaggttacaagagtgtgaatatcacttttgaacttgatgaaaggatt
gataaagtacttaatgagaagtgctctgcctatacagttgaactcggtacagaagtaaatgagttcgcctgtgt
tgtggcagatgctgtcataaaaactttgcaaccagtatctgaattacttacaccactgggcattgatttagatg
agtggaGTATGGCTACATACTACTtatttgatgagtctggtgagtttaaattggcttcacatatgtattgttct
ttctaccctccagatgaggatgaagaagaaggtgattgtgaagaagaagagtttgagccatcaactcaatatga
gtatggtactgaagatgattaccaaggtaaacctttggaatttggtgccacttctgctgctcttcaacctgaag
aagagcaagaagaagattggttagatgatgatagtcaacaaactgttggtcaacaagacggcagtgaggacaat
cagacaactactattcaaacaattgttgaggttcaacctcaattagagatggaacttacaccagttgttcagac
tattgaagtgaatagttttagtggttatttaaaacttactgacaatgtatacattaaaaatgcagacattgtgg
aagaagctaaaaaggtaaaaccaacagtggttgttaatgcagccaatgtttaccttaaacatggaggaggtgtt
gcaggagccttaaataaggctactaacaatgccatgcaagttgaatctgatgattacatagctactaatggacc
acttaaagtgggtggtagttgtgtTTTAAGCGGACACAATCTTgctaaacactgtcttcaTGTTGTCGGCCCAA
ATGTtaacaaaggtgaagacattcaacttcttaagagtgcttatgaaaattttaatcagcacgaagttctactt
gcaccattattatcagctggtatttttggtgctgaccctatacattctttaagagtttgtgtagatactgttcg
cacaaatgtctacttagctgtctttgataaaaatctctatgacaaacttgtttcaagctttttggaaatgaaga
gtgaaaagcaagttgaacaaaagatcgctgagattcctaaagaggaagttaagccatttataactgaaagtaaa
ccttcagttgaacagagaaaacaagatgataagaaaatcaaagcttgtgttgaagaagttacaacaactctgga
agaaactaagttcctcacagaaaacttgttactttatattgacattaatggcaatcttcatccagattctgcca
ctcttgttagtgacattgacatcactttcttaaagaaagatgctccatatatagtgggtgatgttgttcaagag
ggtgttttaactgctgtggttatacctactaaaaaggctggtggcactactgaaatgctagcgaaagctttgag
aaaagtgccaacagacaattatataaccACTTACCCGGGTCAGGGTttaaatggttacactgtagaggaggcaa
agacagtgcttaaaaagtgtaaaagtgccttttacattctaccatctattatctctaatgagaagcaagaaatt
cttggaactgtttcttgGAATTTGCGAGAAATGCTtgcacatgcagaagaaacacgcaaattaatgcctgtctg
tgtggaaactaaagccatagtttcaactatacagcgtaaatataagggtattaaaatacaagagggtgtggttg
attatggtgctagattttacttttacaccagtaaaacaACTGTAGCGTCACTTATCAACACACTTAACGATCTA
Aatgaaactcttgttacaatgccacttggctatgtaacacatggcttaaatttggaagaagctgctcggtatat
gagatctctcaaagtgccagctacagtttctgtttcttcacctgatGCTGTTACAGCGTATAATGGTTatctta
cttcttcttctaaaacacctgaagaacattttattgaaaccatctcacttgctggttcctataaagattggtcc
tattctggacaatctacacaactaggtatagaatttcttaagagaggtgataaaagtgtatattacactagtaa
tcctaccacattccacctagatggtgaagttatcacctttgacaatcttaagacacttctttctttgagagaag
tgaggactattaaggtgtttacaacagtagacaacattaaccTCCACACGCAAGTTGTGGacatgtcaatgaca
tatggacaacagtttggtccaacttatttggatggagctgatgttactaaaataaaacctcataattcacatga
aggtaaaacattttatgttttacctaatgatgacactctacgtgttgaggcttttgagtactaccacacaactg
atcctagttttctgggtaggtacatgtcagcattaaatcacactaaaaagtggaaatacccacaagttaatggt
ttaacttctattaaatgggcagataacaactgttatcttgccactgcattgttaacactccaacaaatagagtt
gaagtttaatccacctgctctacaagatgcttattacagagcaagggctggtgaagctgctaacttttgtgcac
ttatcttagcctactgtaataagacagtaggtgagttaggtgatgttagagaaacaatgagttacttgtttcaa
catgccaatttagattcttgcaaaagagtcttgaacgtggtgtgtaaaacttgtggacaacAGCAGACAACCCT
TAAGGgtgtagaagctgttatgtacatgggcacactttcttatgaacaatttaagaaaggtgttcaGATACCTT
GTACGTGTGGTaaacaagctacaaaatatctagtacaacaggagtcaccttttgttatgatgtcagcaccacct
gctcagtatgaacttaagcatggtacatttacttgtgctagtgagtacactggtaattaccagtgtggtcacta
taaacatataacttctaaagaaactttgtattgcatagacggtgctttacttacaaagtcctcagaatacaaag
gtcctattacggatgttttctacaaagaaaacagttacacaacaaccataaaaccagttacttataaattggat
ggtgttgtttgtacagaaattgaccctaagttggacaattattataagaaagacaattcttatttcacagagca
accaattgatcttgtaccaaaccaaccatatccaAACGCAAGCTTCGATAATTttaagtttgtatgtgataata
tcaaatttgctgatgatttaaaccagttaactggttataagaaacctgcttcaagagagcttaaagttacattt
ttccctgacttaaatggtgatgtggtggctattgattataaacactacacaccctcttttaagaaaggagctaa
attgttacataaacctattgtttggcatgttaacaatgcaactaataaagccacgtataaaccaaatacctggt
gtatacgttgtctttggagcacaaaaccagttgaaacatcaaattcgtttgatgtactgaagtcagaggacgcg
cagggaatggataatcttGCCTGCGAAGATCTAAAAccagtctctgaagaagtagtggaaaatcctaccataca
gaaagacgttcttgagtgtaatgtgaaaactaccgaagttgtaggagacattatacttaaaccagcaaataata
gtttaaaaattacagaagaggttggccacacagatctaatggctgcttatgtagacaattctagtcttactatt
aagaaacctaatgaattatctagagtattaggtttgaaaacccttgctactcatggtttagctgctgttaatag
tgtcccttgggatactatagctaattatgctaagccttttcttaacaaagttgttagtacaactactaacatag
ttacacggtgtttaaaccgtgtttgtactaattatatgccttatttctttactttattgctacaattgtgtact
tttactagaagtacaaattctagaattaaagcatctatgccgactactatagcaaagaatactgttaagagtgt
cggtaaattttgtctagaggcttcatttaattatttgaagtcacctaatttttctaaactgataaatattataa
tttggtttttactattaagtgtttgcctaggttctttaatctaCTCAACCGCTGCTTTAGGtgttttaatgtct
aatttaggcatgccttcttactgtactggttacagagaaggctatttgaactctactaatgtcactattgcaac
ctactgtactggttctataccttgtagtgtttgtcttagtggtttagattctttagacacctatccttctttag
aaactatacaaattaccatttcatcttttaaatgggatttaactgcttttggcttagttgcagagtggtttttg
gcatatattcttttcactaggtttttctatgtacttggattggctgcaatcatgcaattgtttttcagctattt
tgcagtacattttattagtaattcttggcttatgtggttaataattaatcttgtacaaatggccccgatttcag
ctatggttagaatgtacatcttctttgcatcattttattatgtatggaaaagttatgtgcatgttgtagacggt
tgtaattcatcaacttgtatgatgtgttacaaacgtaatagagcaACAAGAGTCGAATGTACAActattgttaa
tggtgttagaaggtccttttatgtctatgctaatggaggtaaaggcttttgcaaactacacaattggaattgtg
ttaattgtgatacattctgtgctggtagtacatttattagtgatgaagttgcgagagacttgtcactacagttt
aaaagaccaataaatcctactgacCAGTCTTCTTACATCGTTGAtagtgttacagtgaagaatggttccatcca
tctttactttgataaagctggtcaaaagacttatgaaagacattctctctctcattttgttaacttagacaacc
tgagagctaataacactaaaggttcattgcctattaatgttatagtttttgatggtaaatcaaaatgtgaagaa
tcatctgcaAAATCAGCGTCTGTTTACtacagtcagcttatgtgtcaacctatactgttactagatcaggcatt
agtgtctgatgttggtgatagtgcggaagttgcagttaaaatgtttgatgcttacgttaatacgttttcatcaa
ctTTTAACGTACCAATGGAAAaactcaaaacactagttgcaactgcagaagctgaacttgcaaagaatgtgtcc
ttagacaatgtcttatctacttttatttcagcagctcggcaagggtttgttgattcagatgtagaaactaaaga
tgttgttgaatgtcttaaattgtcacatcaatctgacatagaagTTACTGGCGATAGTTGTAATaactatatgc
tcacctataacaaagttgaaaacatgacaccccgtgaccttggtgcttgtattgactgTAGTGCGCGTCATATT
AATGCGCAGGTAGCAAAaagtcacaacattgctttgatatggaacgttaaagatttcatgtcattgtctgaaca
actacgaaaacaaatacgtagtgctgctaaaaagaataacttaccttttaagttgacatgtgcaactactagac
aagttgttaatgttgtaacaacaaagatagcacttaagggtggtaaaattgttaataattggttgaagcagtta
attaaagttacacttgtgttcctttttgttgctgctattttctatttaataacacctgttcatgtcatgtctaa
acatactgacttttcaagtgaaatcataggatacaaggctattgatgGTGGTGTCACTCGTGACAtagcatcta
cagatacttgttttgctaacaaacatgctgattttgacacatggtttagccagcgtggtggtagttatactaat
gacaaagcttgcccattgattgctgcagtcataacaagagaagtgggttttgtcgtgcctgGTTTGCCTGGCAC
GATATTACGCACAACTAatggtgactttttgcatttcttacctagagtttttagtgcagttggtaacatctgtt
acacaccatcaaaacttatagagtacactgactttgcaacatcagcttgtgttttggctgctgaatgtacaatt
tttaaagatgcttctggtaagccagtaccatattgttatgataccaatgtactagaaggttctgttgcttatGA
AAGTTTACGCCCTGACACACGTTATGtgctcatggatggctctattattcaatttcctaacacctaccttgaag
gttctgttagagtggtaacaacttttgattctGAGTACTGTAGGCACGGCacttgtgaaagatcagaagctggt
gtttgtgtatctactagtggtagatgggtacttaacaatgattattacagatctttaccaggagttttctgtgg
tgtagatgctgtaaatttacttactaatatgtttacaccactaattcaacctattggtgctttggacatatcag
catctatagtagctggtggtattgtagctatcgtagtaacatgccttgcctactattttatgaggtttagaaga
gcttttggtgaatacagtcatgtagttgcctttaatactttactattccttatgtcattcactgtactctgttt
aacaccagtttactcattcttacctggtgtttattctgttatttacttgtacttgacattttatcttactaatg
atgtttcttttttagcacatattcagtggatggttatgttcacacctttagtacctttctggataacaattgct
tatatcatttgtatttccacaaagcatttctattggttctttagtaattacctaaagagacgtgtagtctttaa
tggtgtttcctttagtacttttgaaGAAGCTGCGCTGTGCACCtttttgttaaataaagaaatgtATCTAAAGT
TGCGTAGTGAtgtgCTATTACCTCTTACGCAATATAATAGatacttagctctttataataagtacaagtatttt
agtggagcaatggatacaactagctacagagaagctgcttgttgtcatctcgcaaaggctctcaatgacttcag
taactcaggttctgatgttctttaccaaccaccacaaacctctatcacctcagctgttttgcagagtggtttta
gaaaaatggcattcccatctggtaaagttgagggttgtatggtacaagtaacttgtggtacaactacacttaac
ggtctttggcttgatgacgtagtttactgtccaagacatgtgatctgcacctctgaagacatgcttaaccctaa
ttatgaagatttactcaTTCGTAAGTCTAATCATAatttcttggtacaggctggtaatgttcaactcagggtta
ttggacattctatgcaaaattgtgtacttaagcttaaggttgatacagccaatcctaagacacctaagtataAG
TTTGTTCGCATTCAACcaggacagactttttcagtgttagcttgttacaatggttcaccatctggtgtttacca
atgtgctatgaggcccaatttcactattaagggttcattccttaatggttcatgtggtagtgttggttttaaca
tagattatgactgtgtctctttttgttacatgcaccatatggaattaccaactggagttcatgctggcacagac
ttagaaggtaacttttatggaccttttgttgacaggcaaacagcacaagcagctggtacggacacaactattac
agttaatgttttagcttggttgtacgctgctgttataaatggagacaGGTGGTTTCTCAATCGATttaccacaa
ctcttaatgactttaaccttgtggctatgaagtacaattatgaacctctaacacaagaccatgttgacatacta
ggacctctttctgctcAAACTGGAATTGCCGTTTTagatatgtgtgcttcattaaaagaattactgcaaaatgg
tatgaatggacgtaccatattgggtagtgctttattagaagatgaatttacaccttttgatgttgttagacaat
gctcaggtgttactttccaaagtgcagtgaaaagaacaatcaagggtacacaccactggttgttactcacaatt
ttgacttcacttttagttttagtccagagtactcaatggtctttgttcttttttttgtatgaaaatgccttttt
accttttgctatgggtattattgctatgtctgcttttgcaatgatgtttgtcaaacataagcatgcatttctct
gtttgtttttgttaccttctcttgccactgtagcttattttaatatggtctaTATGCCTGCTAGTTGGGTGatg
cgtattatgacatggttggatatggttgatactagtttgtctggttttaagctaaaagactgtgttatgtatgc
atcagctgtagtgttactaatccttatgacagcaagaactgtgtatgatgatggtgctaggagagtgtggacac
ttatgaatgtcttgacactcgtttataaagtttattatggtaatgctttagatcaagccatttccatgtgggct
cttataatctctgttacttctaactactcaggtgtagttacaactgtcatgtttttggccagaggtattgtttt
tatgtgtgttgagtattgccctattttcttcataactggtaatacacttcagtgtataatgctagtttattgtt
tcttaggctatttttgtacttgttactttggcctcttttgtttactcaaccgctactttagactgactcttggt
gtttatgattacttagtttctacacaggagtttagatatatgaattcacagggactactcccacccaagaatag
catagatgccttcaaactcaacattaaattgttgggtgttggtggcaaaccttgtatcaaagtagccactgtac
agtctaaaatgtcagatgtaaagtGCACATCAGTAGTCTTACTCTCagttttgcaacaactcagagtagaatca
tcatctaaattgtgggctcaatgtgtccagttacacaatgacattctcttagctaaagatactactgaagcctt
tgaaaaaatggtttcactactttctgttttgctttccatgcagggtgctgtagacataaacaagctttgtgaag
aaatgctggacaacagggcaaccttacaagctatagcctcagagtttagttcccttccatcatatgcagctttt
gctactgctcaagaagcttatgagcaggctgttgctaatggtgattctgaagttgttcttaaaaagttgaagaa
gtctttgaatgtggctaaatctgaatttgaccgtgatgcagccatgcaacgtaagttggaaaagatggctgatc
aagctatgacccaaatgtataaacaggctagatctgaggacaagagggcaaaagttactagtgctatgcagaca
atgcttttcactatgcttagaaagttggataatgatgcactcaacaacattatcaacaatgcaagagatggttg
tgttcccttgaacataatacctcttacaacagcagccaaactaatggttgtcataccagactataacacatata
aaaatacgtgtgatggtacaacatttacttatgcatcagcattgtgggaaatccaacaggttgtagatgcagat
agtaaaattgttcaacttagtgaaattagtatggacaattcacctaatttagcatggcctcttattgtaacagc
tttaagggccaattctgctgtcaaattacagaataATGAGCTTAGTCCTGTTGCaCTACGACAGATGTCTTGTg
ctgccggtactacacaaactgcttgcacTGATGACAATGCGTTAGCTTACTACAAcacaacaaagggaggtagg
tttgtacttgcactgttatccgatttacaggatttgaaatgggctagattccctaagagtgatggaactggtac
tatctatacagaactggaaccaccttgtaggtttgttacagacacacctaaaggtcctaaagtgaagtatttat
actttattaaaggattaaacaacctaaatagaggtatggtacttggtagtttagctgccacagtacgtctacaa
gctggtaatgcaacagaagtgcctgccaattcaactgtattatctttctgtgcttttgctgtagatgctgctaa
agcttacaaagattatCTAGCTAGTGGGGGACAACCAATcactaattgtgttaagatgttgtgtacacacactg
gtactggtcaggcaataacagttacaccggaagccaatatggatcaagaatcctttggtggtgcatcgtgttgt
ctgtactgCCGTTGCCACATAGATCAtccaaatcctaaaggattttgtgacttaaaaggtaagtatgtacaaat
acctacaacttgtgctaatgaccctgtgggttttacacttaaaaacacagtCTGTACCGTCTGCGGTATGtgga
aaggttatggctgtagttgtgatcaACTCCGCGAACCCATGCTTcagtcagctgatgcacaatcgtttttaaac
gggtttgcggtgtaagtgCAGCCCGTCTTACACCGTGCGGCACAGGCACTAGTACtgatgtcgtatacagggct
tttgacatctacaatgataaagtagctggttttgctaaattcctaaaaactaattgttgtcgcttccaagaaaa
ggacgaagatgacaatttaattgattcttactttgtagttaagagacacactttctctaactaccaacatgaag
aaacaatttataatttacttaaggattgtccagctgttgctaaacatgacttctttaagtttagaatagacggt
gacatggtaccacatatatcacgtcaacgtcttactaaatacacaatggcagacctcgtctatgctttaaggca
ttttgatgaaggtaattgtgacacattaaaagaaatacttgtcacatacaattgttgtgatgatgattatttca
ataaaaaggactggtatgattttgtagaaaacccagatatattacgCGTATACGCCAACTTAGGTGAACGTGTA
CGccaagctttgttaaaaacagtacaattctgtGATGCCATGCGAAATGCTGGTattgttggtgtactgacatt
agataatcaagatctcaatggtaactggtatgatttcggtgatttcatacaaaccacgccaggtagtggagttc
ctgttgtagattcttattattcattgttaatgcctatattaaccttgaccagggctttaactgcagagtcacat
gttgacactgacttaacaaagccttacattaagtgggatttgttaaaatatgacttcacggaagagaggttaaa
actctttgaccgttattttaaatattgggatcagacataccacccaaattgtgttaactgtttggatgacagat
gcattctgcattgtgcaaactttaatgttttattctCTACAGTGTTCCCACCTAcaagttttggaccactagtg
agaaaaatatttgttgatggtgttccatttgtagtttcaactggataccacttcagagagctaggtgttgtaca
taatcaggatgtaaacttacaTAGCTCTAGACTTAGTTTtaaggaattacttgtgtatgctgctgaccctgcta
tgcacgctgcttctggtaatctattactagataaacgcactacgtgcttttcagtagctgcacttactaacaat
gttgcttttcaaactgtcaaacccggtaattttaacaaagacttctatgactttgctgtgtctaagggtttctt
taaggaaggaagttctgttgaattaaaacacttcttctttgctcaggatggtaatgctgctATCAGCGATTATG
ACTACTATCGTTATAatctaccaacaatgtgtgatatcagacaactactatttgtagttgaagttgttgataag
tactttgattgTTACGATGGTGGCTGTATtaatgctaaccaagtcatcgtcaacaacctagacaaatcagctgg
ttttccatttaataaatggGGTAAGGCTAGACTTTATtatgattcaatgagttatgaggatcaagatgcacttt
tcgcatatacaaaacgtaatgtcatccctactataactcaaatgaatcttaagtatgccattagtgcaaagaat
agagctcgcaccgtagctggtgtctctatctgtagtactatgaccaatagacagtttcatcaaaaattattgaa
atcAATAGCCGCCACTAGAGGAgctactgtagtaattggaacaagcaaattctatggtggttggcacaacatgt
taaaaactgtttatagtgatgtagaaaaccctcaccttatgggttgggattatcctaaatgtgatagagccatg
cctaacatgcttagaattatggcctcacttgttcttgctcgcaaacatacAACGTGTTGTAGCTTGTCACACCG
TTTctatagattagctaatgagtgtgctcaagtattgagtgaaatggtcatgtgTGGCGGTTCACTATATGTTA
aaccaggtggaacctcatcaggagatgccacaactgcttatgctaatagtgtttttaacatttgtcaagctgtc
acggccaatgttaatgcacttttatctactgatggtaacAAAATTGCCGATAAGTATGTCcgcaatttacaaca
cagactttatgagtgtctctatagaaatagagatgttgacacagactttgtgaatgagtttTACGCATATTTGC
GTAAACatttctcaatgATGATACTCTCTGACGATGCtgttgtgtgtttcaATAGCACTTATGCATCTCAaggt
ctagtggctagcataaagaactttaagtcagttctttattatcaaaacaatgtttttatgtctgaagcaaaatg
ttggactgagactgaccttactaaaggacctcatgaattttgctctcaacatacaatgctagttaaacagggtg
atgattatgtgtaccttccttacccagatccatcaagaatcctaggggccggctgttttgtagatgatatcgta
aaaacagatggtacacttATGATTGAACGGTTCGTGTCTTtagctatagatgcttacccacttactaaacatcc
taatcaggagtatgctgatgtctttcatttgtacttacaatacataagaaagctacatgatgagttaacaggac
acatgttagacatgtattctgttatgcttactaatgataacacttcaaggtattgggaacctgagttttatgag
gctatgtacacaccgcatacagtcttacaggctgttggggcttgtgttctttgcaattcacagacttcattaag
atgtggtgcttGCATACGTAGACCATTCTtatgttgtaaatgctgttacgaccatgtcatatcaacatcacata
aattagtcttgtcTGTTAATCCGTATGTTTGCAatgctccaggttgtgatgtcacagatgtgactcaactttac
ttagGAGGTATGAGCTATTATTgtaaatcacataaaccacccattagttttccattgtgtgctaatggacaagt
ttttggtttatataaaaatacATGTGTTGGTAGCGATAATGttactgactttaatgcaattgcaacatgtgact
ggacaaatgctggtgattacattttagctaacacctgtactgaaagactcaagctttttgcaGCAGAAACGCTC
AAAGCTactgaggagacatttaaactgtcttatGGTATTGCTACTGTACGTGAAGTGCTGTCTgacagagaatt
acatctttcatgggaagttggtaaacctagaCCACCACTTAACCGAAATTATGTCTttactGGTTATCGTGTAA
CTAAAaacagtaaagtacaaataggagagtacacctttgaaaaaggtgactatggtgatgctGTTGTTTACCGA
GGTACAAcaacttacaaattaaatgttggtgattattttgtgctgacatcacatacagtaatgccattaagtgc
acctacactaGTGCCACAAGAGCACTATgttagaattactggcttatacccaacactcaatatctcagatgagt
tttctagcaatgttgcaaattatcaaaaggttggtatgcaaaagtattctacactccagggaccacctggtact
ggtaagagtcattttgctattggcctagctctctactacccttctgctcgcatagtgtatacagcttGCTCTCA
TGCCGCTGTTGATGcactatgtgagaaggcattaaaatatttgcctatagataaatgtagtagaattatacctg
cacgtgctcgtgtagagtgttttgataaattcaaagtgaattcaacattagaacagtatgtcttttgtactgta
aatGCATTGCCTGAGACGACAGCagatatagttgtctttgatgaaatttcaatggccacaaattatgatttgag
tgttgtcaATGCCAGATTACGTGCTAAGCActatgTGTACATTGGCGACCCTGctcaattacctgcaccacgca
cattgctaactaagggcacactagaaccagaatatttcaattcagtgtgtagacttatgaaaactataggtcca
gacatgttcctcggaacttgtcggcgttgtcctgctgaaattgttgacactgtgagtgctttggtttatgataa
taagcttaaagcacataaagacaaatcagctcaatgctttaaaatgttttaTAAGGGTGTTATCACGCATgatg
tttcatctgcaattaacaGGCCACAAATAGGCGTGGtaagagaATTCCTTACACGTAACCCTGCTTGGAGAaaa
gctgtctttatttcaccttataattcacagaatgctgtagcctcaaagattttgggactaccaactcaaactgt
tgattcatcacagggctcagaatatgactatgtcatattcactcaaaccactgaaacagctcactcttgtaatg
taaacagatttaatgttgctattaccagagcaaaagtaggcatactttgcataatgtctgatagagacctttat
gacaagttgcaatttacaagtcttgaaATTCCACGTAGGAATGTGGCAACtttacaagctgaaaatgtaacagg
actctttaaagattgtagtaaggtaatcactgggttacatcctacacaggcacctacacacctcagtgttgaca
ctaaattcaaaactgaaggtttatgtgttgacatacctggcatacctaaggacatgacctatagaagactcatC
TCTATGATGGGTTTTAAaatgaattatcaagttaatggttaccctaacatgtttatcacccgcgaagaagctat
aagACATGTACGTGCATGGATTggcttcgatgtcgaggggtgtcatgctactagagaagctgttggtaccaatt
tacctttacagctaggtttttctaCAGGTGTTAACCTAGTTGCtgtacctacaggttatgttgatacacctaat
aatacagatttttccagagttagtgctaaaccaccgcctggagatcaatttaaacacctcataccacttatgta
caaaggacttccttGGAATGTAGTGCGTATAAAGATtgtacaaatgttaagtgacacacttaaaaatctCTCTG
ACAGAGTCGTATTTGtcttatgggcacatggctttgagttgacatctatgaagtattttgtgaaaataGGACCT
GAGCGCACCTGTTGtctatgtgatagacgtgccacatgcttttccactgcttcagacacttatgcctgttggca
tcattctattggatttgaTTACGTCTATAATCCGTTTATGAttgatgttcaacaatggggttttacaggtaacc
tacaaagcaaccatgatctgtattgtcaagtccatggtaatgcacatgtagctagttgtgatgcaatcatgact
aggtgtctagctgtccacgagtgctttgttaagcgtgttgactggactattgaatatcctataattggtgatga
actgaagatTAATGCGGCTTGTAGAAAGGTtcaacacatggttgttaaagctgcattattagcagacaaattcc
cagttcttcacgacattggtaaccctaaagctattaagtgtgtacctcaagctgatgtagaatggaagttctat
gatgcacagccttgtagtgacaaagcttataaaatagaagaattattctattcttatgccacacattctgacaa
attcacagatggtgtatgcctattttggaATTGCAATGTCGATAGATAtcctgctaattccattgtttgtagat
ttgACACTAGAGTGCTATCTAaccttaacttgcctggttgtgatggtggcagtttgtatgtaaataaacatgca
ttccacacaccagcttttgataaaagtgcttttgttaatttaaaacaattaccatttttctattactctgacag
tccatgtgagtctcatggaaaacaagtagtgtcagatatagattatgtaccactaaagtCTGCTACGTGTATAA
CACgttgcaatttaggtggtgctgtctgtagacatcatgctaatgagtacagattgtatctcgatgcttataac
atgatgatctcagctggctttagcttgtgggtttacaaacaatttgatacttataacctctggaacacttttac
aagacttcagagtttagaaaatgtggcttttaatgttgtaaataagggacactttgatggacaacagggtgaag
taccagtttctatcattaataacactgtttacacaaaagttgatggtgttgatgtagaattgtttgaaaataaa
acaacattacctgttaatgtagcatttGAGCTTTGGGCTAAGCGCAACATTAAaccagtaccagaggtgaaaat
actcaataatttgggtgtggacattgctgctaatactgtgatctgggactacaaaagagatgctccagcacata
tatctactattggtgtttgttctatgactgacatagccaagaaaccaactgaaacgatttgtgcaccactcact
gtcttttttgatggtagagttgATGGTCAAGTAGACTTATttaGAAATGCCCGTAATGGTGttcttattacaga
aggtagtgttaaaggtttacaaccatctgtaggtcccaaacaagctagtcttaatggagtcacattaattggag
aagccgtaaaaacacagttcaattattataagaaagttgatggtgttgtccaacaattacctgaaacttacttt
actcagagtagaaatttacaagaatttaaacccaggagtcaaatggaaattgatttcttagaattagctatgga
tgaattcattgaacggtataaattagaaggctatgCCTTCGAACATATCGTTTATGgagattttaGTCATAGTC
AGTTAGGTGGTttacatctactgATTGGACTAGCTAAACGTTttaaggaatcaccttttgaattagaagatttt
attcctatggacagtacagttaaaaactatttcataacagatgcgcaaacaggttcatctaagtgtgtgtgttc
tgttattgatttattacttgatgattttgttgaaataataaaatcccaagatttatctgtagtttctaaggttg
tcaaagtgactattgactatacagaaatttcatttatgctttggtgtaaagatggccatgtagaaacattttac
ccaaaatTACAATCTAGTCAAGCGTGGCAACCGGGTGTTgctatgcctaatctttacaaaatgcaaagaatgct
attagaaaagtgtgaccttcaaaattatggtgatagtgcaacattacctaaaggcataatgatgaatgtcgcaa
aatatactcaactgtgtcaatatttaaacacattaacattagctgtaccctataatatgagagttatacatttt
ggtgctggttctgataaaGGAGTTGCACCAGGTACAgctgttttaagacagtggttgcctacgggtacgctgct
tgtcgattcagatcttaatgactttgtctctgatgcagattcaactttgattggtgattgtgcaactgtacata
cagctaataaatgggatctcattattagtgatatgTACGACCCTAAGACTAAAaatgttacaaaagaaaatgac
tctaaagagggttttttcacttacatttgtgggtttatacaacaaaagctagctcttggaggttccgtggctat
aaagataacagaacattcttggaatgctgatctttataagctcaTGGGACACTTCGCATGGTGGACagcctttg
ttACTAATGTGAATGCGTCATCAtctgaagcatttttaattggatgtaattATCTTGGCAAACCACGCGAACAA
ATAGAtggttatgtcatgcatgcaaattacatattttggaggaatacaaatccaattcagttgtcttcctattc
tttatttgacatgagtaaatttccccttaaattaaggggtactgctgttatgtctttaaaagaaggtcaaatca
atgatatgattttatctcttcttagtaaaggtagacttataattagagaaaacaacagagttgttatttctagt
gatgttcttgttaacaactaaacgaacaatgtttgtttttcttgttttattgccactagtctctagtcagtgtg
ttaatcttacaaccagaactcaattaccccctgcatacactaattctttcacacgtggtgtttattaccctgac
aaagttttcagatcctcagttttacattcaactcaggacttgttcttacctttcttttccaatgttacttggtt
ccatgctatacatgtctctgggaccaatggtactaagaggtttgataaccctgtcctaccatttaatgatggtg
tttattttgcttccactgagaagtctaacataataagaggctggatttttggtactactttagattcgaagacc
cagtccctacttaTTGTTAATAACGCTACTAatgttgttattaaagtctgtgaatttcaattttgtaatgatcc
atttttgggtgtttattaccacaaaaacaacaaaagttggatggaaagtgagttcagagtttattctagtgcga
ataattgcacttttgaatatgtctctcagccttttcttatggaccttgaaggaaaacagggtaatttcaaaaat
cttagggaatttgtgtttaagaatattgatggttattttaaaataTATTCTAAGCACACGCCTATTAATTTAGT
GCGTGATCTCCCtcagggtttttcggctttagaaccattggtagatttgccaataggtattaacatcactaggt
ttcaaactttacttgctttacatagaagttatttgactcctggtgattcttcttcaggttggacagctggtgct
gcagcttattatgtgggttatcttcaacctaggacttttctattaaaatataatgaaaatggaaccattacaga
tgctgtagactgtgcacttgaccctctctcagaaacAAAGTGTACGTTGAAATCcttcactgtagaaaaaggaa
tctatcaaacttctaactttagagtccaaccaacagaatctattgttagatttcctaatattacaaacttgtgc
ccttttggtgaagtttttaacgccaccagatttgcatctgtttatgcttggaacaggaagagaatcagcaactg
tgttgctgattattctgtcctatataattccgcatcattttccacttttaagtgttatggagtgtctcctacta
aattaaatgatctctgctttactaatgtctatgcagattcatttgtaattagaggtgatgaagtcagacaaatc
gctccagggcaaactggaaagattgctgattataattataaattaccagatgattttacaggctgcgttatagc
ttggaattctaacaatcttgattctaaggttggtggtaattataattacctgtatagattgtttaggaagtcta
atctcaaaccttttgagagagatatttcaactgaaatctATCAGGCCGGTAGCACACCTTGTaatggtgttgaa
ggttttaattgttactttcctttacaatcatatggtttccaacccactaatggtgttggttaccaaccatacag
agtagtagtactttcttttgaacttctacatgcaccagcaactgtttgtggacctaaaaagtctactaatttgg
ttaaaaacaaatgtgtcaatttcaacttcaatggtttaacaggcacaggtGTTCTTACTGAGTCTAACAaaaag
tttctgcctttccaacaatttggcagagacattgctgacactactgatgctgtccgtgatccacagacacttga
gattcttgacattacaccatgttcttttggtggtgtcagtgttataacaccaggaacaaatacttctaaccagg
ttgctgttctttatcaggatgttaactgcacagaagtccctgttgctattcatgcagatcaacttactcctact
tggcgtgtttattctacaggttctaatgtttttcaaacacgtgcaggctgtttaataggggctgaacatgtcaa
caactcatatgagtgtgacatacccattGGTGCAGGTATATGCGCTAGTTATCAGACTCagactaattctcctc
ggcgggcacgtagtgtagctagtcaatccatcattgcctacactatgtcacttggtgcagaaaattcagttgct
tactctaataactctattgccatacccacaaattttactattagtgttaccacagaaattctaccagtgtctat
gaccaagacatcagtagattgtacaatgtacatttgtggtgattcaactgaatgcagcaatcttttgttgcaat
atggcagtttttgtacacaaTTAAACCGTGCTTTAACTggaatagctgttgaacaagacaaaaacacccaag
aagtttttgcacaagtcaaacaaatttacaaaacaccaccaattaaagattttggtggttttaatttttcacaa
atattaccagatccatcaaaaccaagcaagaggtcatttattgaagatctacttttcaacaaagtgacacttgc
agatgctggcttcatcaaacaatatggtgattgccttggtgatattgctgctagagacctcatttgtgCACAAA
AGTTTAACGGCCTTACTGTTttgccacctttgctcacagatgaaatgattgctcaatacacttctgcactgtta
gcgggtacaatcacttcTGGTTGGACCTTTGGTGCaggtgctgcattacaaataccatttgctatgcaaatggc
ttataggtttaatggtattggagttacacagaatgttctctatgagaaccaaaaattgattgccaaccaattta
atagtgctattggcaaaattcaagactcactttcttccacagcaagtgcacttggaaaacttcaagatgtggtc
aaccaaaatgcacaagctttaaacacgcttgttaaacaacttagctccaattttggtgcaatttcaagtgtttt
aaatgatatcctttcacgtcttgacaaagttgaggctgaagtgcaaattgataggttgatcacaggcagacttc
aaagtttgcagacatatgtgactcaacaattaattagagctgcagaaatcagagcttctgctaatcttgctgct
actaaaatgtcagagtgtgtacttggacaatcaaaaagagttgatttttgtggaaagggctatcatcttatgtc
cttccctcagtcagcacctcatggtgtagtcttcttgcatgtgacttatgtccctgcacaagaaaagaacttca
caactgctcctgccatttgtcatgatggaaaagcacACTTTCCTCGTGAAGGTGtctttgtttcaaatggcaca
cactggtttgtaacacaaaggaatttttatgaaccacaaatcattactacagacaacacatttgtgtctggtaa
ctgtgatgttgtaataggaattgtcaacaacacagtttatgatcctttgcaacctgaattagactcattcaagg
aggagttagataaatattttaagaatcatacatcaccagatgttgatttaggtgacatctctggcattaatgct
tcagttgtaaacattcaaaaagAAATTGACCGCCTCAATGAGGTTGccaagaatttaaatgaatctctcatcga
tctccaagaacttggaaagtatgagcagtatataaaatggccatggtacatttggctaggttttatagctggct
tgattgccatagtaatggtgacaattatgctttgctgtatgaccagttgctgtagttgtctcaagggctgttgt
tcttgtggatcctgctgcaaatttgatgaAGACGACTCTGAGCCAGTgctcaaaggagtcaaattacattacac
ataaacgaacttatggatttgtttatgagaatcttcacaattggaactgtaactttgaagcaaggtgaaatcaa
ggatgctactccttcagATTTTGTTCGCGCTACTGCAACGATaccgatacaagcctcactcccTTTCGGATGGC
TTATTGTTGGCGTTgcacttcttgctgtttttcagagcgcttccaaaatcataaccctcaaaaagagatggcaa
ctagcactctccaagggtgttcactttgtttgcaacttgctgttgttgtttgtaacagtttactcacacctttt
gctcgttgctgctggccttgaagccccttttctctatctttatgctttagtctacttcttgcagagtataaact
ttgtaagaataataatgaggctttggctttgctggaaatgccgttccaaaaacccattactttatgatgccaac
tattttctttgctggcatactaattgttacgactattgtataccttacaatagtgtaacttcttcaattgtcat
tacttcaggtgatggcacaacaagtcctatttctgaacatgactaccagattggtggttatactgaaaaatggg
aatctggagtaaaagactgtgttgtattacacagttacttcacttcagactattaccagctgtactcaactcaa
ttgagtacagacactggtgttgaacatgttaccttcttcatctacaataaaattgttgatgagcctgaagaaca
tgtccaaatTCACACAATCGACGGTTCATCcggagttgttaatccagtaatggaaccaattTATGATGAACCGA
CGACGActactagcgtgcctttgtaagcACAAGCTGATGAGTACGAACTTATGtactcattcgtttcggaaGAG
ACAGGTACGTTAATAGTtAATAGCGTACTTCTTTTTcttgctttcgtggtattcttgctagttacactagccat
ccttactGCGCTTCGATTGTGTGCGTACTgctgcaatattgttaacgtgagtcttgtaaaaccttcttTTTACG
TTTACTCTCGTGTtaaaaatctgaattcttctagagttcctgatcttctggtctaaacgaactaaatattatat
tagtttttctgtttggaactttaattttagccatGGCAGATTCCAACGGTACTATTACCGTTGAAGAGCTtaaa
aagctccttgaacaatggaacctagtaataggtttcctattccttacatggatttgtcttctacaatttgccta
tgccaacaggaataggtttttgtatataattaagttaattttcctctggctgttatggccagtaactttagctt
gttttgtgcttgctgctgtttacagaataaattggatcaccggtggaattgctatcgcaatggcttgtcttgta
ggcttgatgtggctcagctacttcattgcttctttcagactgtttGCGCGTACGCGTTCCATGTggtcattcaa
tccagaaactaacattcttctcaacgtgccactccatggcacTATTCTGACCAGACCGCTTctagaAAGTGAAC
TCGTAATCGGAGCTGTGATCCttcgtggacatcttcgtattgctggacaccatctaggacgctgtgacatcaag
gacctgcctaaagaaatcaCTGTTGCTACATCACGAACGCTTTcttattaCAAATTGGGAGCTTCGCAgcgtgt
agcaggtgactcaggttTTGCTGCATACAGTCGCTACAGgattggcaactataaattaaacacagaccattcca
gtagcagtgacaatattgctttgcttgtacagtaagtgacaacagatgtttcatctcgttgactttcaggttac
tatagcagagatattactaattattatgaggacttttaaagtttccatttggaatcttgattacatcataaacc
tcataattaaaaatttatctaagtcactaactgagaataaatattctcaattagatgaagagcaaccaatGGAG
ATTGATTAAACGAAcatgaaaattattcttttcttggcactGATAACACTCGCTACTTGTGAGCTTtatcacta
ccaagagtgtgttagaggtacaacagtacttttaaaagaaccttgctcttctggaacaTACGAGGGCAATTCAC
CAtttcatcctctagctgataacaaatttgcactgacttgctttagcactcaatttgcttttgcttgTCCTGAC
GGCGTAAAACACGTCTATCAGTTACGTGCCAGatcagtttcacctaaactgttcatcagacaagaggaagttca
agaactttactctccaatttttcttattgttgcggcaatagtgtttataacactttgcttcacactcaaaagaa
agacagaatgattgaactttcattaattgacttctatttgtgctttttagcctttctgctattccttgttttaa
ttatgcttattatcttttggttctcacttgaactgcaagatcataatgaaacttgtcacgcctaaacgaacatg
aaatttcttgttttcttaggaatcatcacaactgtagctgcatttcaccaagaatgtagtttacagtcatgtac
tcaacatcaaccatatgtagttgatgacccgtgtcctattcacttctattctaaatggtatattagagtaggag
ctagaaaatcagcacctttaattgaattgtgcgtggatgaggctggttctaaatcacccattcagtacatcgAT
ATCGGTAATTATACAGTTtcctgtttaccttttacaattaattgccaggaacctaaattgggtagtcttgtagt
gcgttgttcgttctatgaagactttTTAGAGTATCATGACGTTCGTgttgttttagatttcatctaaacgaaca
aactaaaatgtctgataatggaccccaaaatcagcgaaatgcaccccgCATTACGTTTGGTGGACCctcagatt
caactggcagtaaccagaatggagaacgcagtggggcgcgatcaaaacaacgtcggccccaaggtttacccaat
aatactgcGTCTTGGTTCACCGCTCTCACTCAACATggcaaggaagaccttaaattccctcgaggacaaggcgt
tccaattaacaccaatagcagtccagatgacCAAATTGGCTACTACCGAAGAGCTACCagacgaattcgtggtg
gtgacggtaaaatgaaagatctcagtccaagatggtatttctactacctaggaactgggccagaagctggactt
ccctatggtgctaacaaagacggcaTCATATGGGTTGCAACTGAgggagccttgaatacaccaaaagaTCACAT
TGGCACCCGCAATcctgctaacaatgctgcaatcgtgctacaacttcctcaaggaacaacattgccaaaaggct
tctacgcagaagggagcagaGGCGGCAGTCAAGCCTCTTctcgttcctcatCACGTAGTCGCAACAGTTCaaga
aattcaactccaggcagcagtaggggaacttctcctgctagaatggctggcaatggcggtgatgctgctcttgc
tttgctgctgcttgacagattgaaccagcttgagagcaaaatgtctggtaaaggccaacaacaacaaggccaaa
ctgtcactaagaaatctgctgctgaggcttctaagaagcctcggcaaaaacgtactgccactaaagcatacaat
gtaacacaagctttcggcagacgtggtccagaacaaacccaaggaaattttggggaccaggaactaatcagaca
aggaactgattacaaacattggccgcaaattgcacaatttgcccccagcGCTTCAGCGTTCTTCGGAATGtcgc
gcattggcatggaagtcacaccttcgggaacgtggttgacctacacaggtgccatcaaattggatgacaaagat
ccaaatttcaaagatcaagtcattttgctgaataagcatattgacgcatacaaaacattcccaccaacagagcc
taaaaaggacaaaaagaagaaggctgatgaaactcaaGCCTTACCGCAGAGACAGaagaaacagcaaactgtga
ctcttcttcctgctgcagatttggatgatttctccaaacaattgcaacaatccatgagcagtgctgactcaact
caggcctaaactcatgcagaccacacaaggcagatgggctatataaacgtTTTCGCTTTTCCGTTTACGATATa
tagtctactcttgtgcaGAATGAATTCTCGTAACTACATAGCACaagtagatgtagttaactttaatctcacat
agcaatctttaatcagtgtgtaacattagggaggacttgaaagagccaccacattttcaccgaggccacgcgga
GTACGATCGAGTGTACAGTGAACaatgctagggagagctgcctatatggaagagccctaatgtgtaaaattaat
tttagtagtgctatccccatgtgattttaatagcttcttaggagaatgacaaaaaaaaaaaaaaaaaaaaaaaa
aaaaaaaaa

In various embodiments, the primers are selected from SEQ ID NOS: 1-559.

SEQ ID NO: 1
TTATACCTTCCCAGGTAA
SEQ ID NO: 2
GTCACTCGGCTGCATGCT
SEQ ID NO: 3
GCTTAGTGCACTCACGCA
SEQ ID NO: 4
CTTAGTGCACTCACGCAG
SEQ ID NO: 5
AGGCTGCTTACGGTTTCG
SEQ ID NO: 6
GGCTGCTTACGGTTTCGT
SEQ ID NO: 7
TTTTACAGGTTCGCGACG
SEQ ID NO: 8
TTTACAGGTTCGCGACGT
SEQ ID NO: 9
TTACAGGTTCGCGACGTG
SEQ ID NO: 10
TACAGGTTCGCGACGTGC
SEQ ID NO: 11
ACAGGTTCGCGACGTGCT
SEQ ID NO: 12
GGTCTTATCAGAGGCACG
SEQ ID NO: 13
GTCTTATCAGAGGCACGT
SEQ ID NO: 14
CATCAAACGTTCGGATGC
SEQ ID NO: 15
ATCAAACGTTCGGATGCT
SEQ ID NO: 16
TCAAACGTTCGGATGCTC
SEQ ID NO: 17
CAAACGTTCGGATGCTCG
SEQ ID NO: 18
AAACGTTCGGATGCTCGA
SEQ ID NO: 19
AACGTTCGGATGCTCGAA
SEQ ID NO: 20
ACGTTCGGATGCTCGAAC
SEQ ID NO: 21
CGTTCGGATGCTCGAACT
SEQ ID NO: 22
GTTCGGATGCTCGAACTG
SEQ ID NO: 23
TGGTAGCAGAACTCGAAG
SEQ ID NO: 24
GGTAGCAGAACTCGAAGG
SEQ ID NO: 25
AACTCGAAGGCATTCAGT
SEQ ID NO: 26
ACTCGAAGGCATTCAGTA
SEQ ID NO: 27
CTCGAAGGCATTCAGTAC
SEQ ID NO: 28
TCGAAGGCATTCAGTACG
SEQ ID NO: 29
CGAAGGCATTCAGTACGG
SEQ ID NO: 30
GAAGGCATTCAGTACGGT
SEQ ID NO: 31
AAGGCATTCAGTACGGTC
SEQ ID NO: 32
AGGCATTCAGTACGGTCG
SEQ ID NO: 33
GGCATTCAGTACGGTCGT
SEQ ID NO: 34
GCATTCAGTACGGTCGTA
SEQ ID NO: 35
CATTCAGTACGGTCGTAG
SEQ ID NO: 36
GGTCGTAGTGGTGAGACA
SEQ ID NO: 37
CCCTCATGTGGGCGAAAT
SEQ ID NO: 38
CCTCATGTGGGCGAAATA
SEQ ID NO: 39
CTCATGTGGGCGAAATAC
SEQ ID NO: 40
TCATGTGGGCGAAATACC
SEQ ID NO: 41
CATGTGGGCGAAATACCA
SEQ ID NO: 42
TGGGCGAAATACCAGTGG
SEQ ID NO: 43
GGGCGAAATACCAGTGGC
SEQ ID NO: 44
CAGTGGCTTACCGCAAGG
SEQ ID NO: 45
AGTGGCTTACCGCAAGGT
SEQ ID NO: 46
GTGGCTTACCGCAAGGTT
SEQ ID NO: 47
TGGCTTACCGCAAGGTTC
SEQ ID NO: 48
GGCTTACCGCAAGGTTCT
SEQ ID NO: 49
GCTTACCGCAAGGTTCTT
SEQ ID NO: 50
CTTACCGCAAGGTTCTTC
SEQ ID NO: 51
TTACCGCAAGGTTCTTCT
SEQ ID NO: 52
CTTCTTCGTAAGAACGGT
SEQ ID NO: 53
CATGCGTGAGCTTAACGG
SEQ ID NO: 54
ATGCGTGAGCTTAACGGA
SEQ ID NO: 55
TGCGTGAGCTTAACGGAG
SEQ ID NO: 56
GCGTGAGCTTAACGGAGG
SEQ ID NO: 57
CGTGAGCTTAACGGAGGG
SEQ ID NO: 58
GTGAGCTTAACGGAGGGG
SEQ ID NO: 59
GGGCATACACTCGCTATG
SEQ ID NO: 60
GGCATACACTCGCTATGT
SEQ ID NO: 61
GCATACACTCGCTATGTC
SEQ ID NO: 62
CATACACTCGCTATGTCG
SEQ ID NO: 63
ATACACTCGCTATGTCGA
SEQ ID NO: 64
TACACTCGCTATGTCGAT
SEQ ID NO: 65
ACACTCGCTATGTCGATA
SEQ ID NO: 66
CACTCGCTATGTCGATAA
SEQ ID NO: 67
TGGCTACCCTCTTGAGTG
SEQ ID NO: 68
ATGGGTAGAATTCGATCT
SEQ ID NO: 69
CAGTTGCGTCACCAAATG
SEQ ID NO: 70
TGAGCATAGTCTTGCCGA
SEQ ID NO: 71
GAGCATAGTCTTGCCGAA
SEQ ID NO: 72
AGCATAGTCTTGCCGAAT
SEQ ID NO: 73
GCATAGTCTTGCCGAATA
SEQ ID NO: 74
CATAGTCTTGCCGAATAC
SEQ ID NO: 75
ATAGTCTTGCCGAATACC
SEQ ID NO: 76
TAGTCTTGCCGAATACCA
SEQ ID NO: 77
AGTCTTGCCGAATACCAT
SEQ ID NO: 78
GTCTTGCCGAATACCATA
SEQ ID NO: 79
TCTTGCCGAATACCATAA
SEQ ID NO: 80
ATTCTTCGTAAGGGTGGT
SEQ ID NO: 81
TTCTTCGTAAGGGTGGTC
SEQ ID NO: 82
TTCCACGTGCTAGCGCTA
SEQ ID NO: 83
GGCTGCTCGTGTTGTACG
SEQ ID NO: 84
GCTGCTCGTGTTGTACGA
SEQ ID NO: 85
CTGCTCGTGTTGTACGAT
SEQ ID NO: 86
TGCTCGTGTTGTACGATC
SEQ ID NO: 87
GCTCGTGTTGTACGATCA
SEQ ID NO: 88
CTCGTGTTGTACGATCAA
SEQ ID NO: 89
TCGTGTTGTACGATCAAT
SEQ ID NO: 90
CGTGTTGTACGATCAATT
SEQ ID NO: 91
GTGTTGTACGATCAATTT
SEQ ID NO: 92
TGTTGTACGATCAATTTT
SEQ ID NO: 93
GTTGTACGATCAATTTTC
SEQ ID NO: 94
TTGTACGATCAATTTTCT
SEQ ID NO: 95
TTGACTTCGCAGTGGCTA
SEQ ID NO: 96
TGACTTCGCAGTGGCTAA
SEQ ID NO: 97
GACTTCGCAGTGGCTAAC
SEQ ID NO: 98
ACTTCGCAGTGGCTAACT
SEQ ID NO: 99
CTTCGCAGTGGCTAACTA
SEQ ID NO: 100
AAACTCAAACCCGTCCTT
SEQ ID NO: 101
AACTCAAACCCGTCCTTG
SEQ ID NO: 102
TCTTAGAGACGGTTGGGA
SEQ ID NO: 103
CTTAGAGACGGTTGGGAA
SEQ ID NO: 104
TTAGAGACGGTTGGGAAA
SEQ ID NO: 105
GTATGGCTACATACTACT
SEQ ID NO: 106
TTTAAGCGGACACAATCT
SEQ ID NO: 107
TTAAGCGGACACAATCTT
SEQ ID NO: 108
TGTTGTCGGCCCAAATGT
SEQ ID NO: 109
ACTTACCCGGGTCAGGGT
SEQ ID NO: 110
GAATTTGCGAGAAATGCT
SEQ ID NO: 111
ACTGTAGCGTCACTTATC
SEQ ID NO: 112
CTGTAGCGTCACTTATCA
SEQ ID NO: 113
ACACACTTAACGATCTAA
SEQ ID NO: 114
GCTGTTACAGCGTATAAT
SEQ ID NO: 115
CTGTTACAGCGTATAATG
SEQ ID NO: 116
TGTTACAGCGTATAATGG
SEQ ID NO: 117
GTTACAGCGTATAATGGT
SEQ ID NO: 118
TTACAGCGTATAATGGTT
SEQ ID NO: 119
TCCACACGCAAGTTGTGG
SEQ ID NO: 120
AGCAGACAACCCTTAAGG
SEQ ID NO: 121
GATACCTTGTACGTGTGG
SEQ ID NO: 122
ATACCTTGTACGTGTGGT
SEQ ID NO: 123
AACGCAAGCTTCGATAAT
SEQ ID NO: 124
ACGCAAGCTTCGATAATT
SEQ ID NO: 125
GCCTGCGAAGATCTAAAA
SEQ ID NO: 126
CTCAACCGCTGCTTTAGG
SEQ ID NO: 127
ACAAGAGTCGAATGTACA
SEQ ID NO: 128
CAAGAGTCGAATGTACAA
SEQ ID NO: 129
CAGTCTTCTTACATCGTT
SEQ ID NO: 130
AGTCTTCTTACATCGTTG
SEQ ID NO: 131
GTCTTCTTACATCGTTGA
SEQ ID NO: 132
AAATCAGCGTCTGTTTAC
SEQ ID NO: 133
TTTAACGTACCAATGGAA
SEQ ID NO: 134
TTAACGTACCAATGGAAA
SEQ ID NO: 135
TTACTGGCGATAGTTGTA
SEQ ID NO: 136
TACTGGCGATAGTTGTAA
SEQ ID NO: 137
ACTGGCGATAGTTGTAAT
SEQ ID NO: 138
TAGTGCGCGTCATATTAA
SEQ ID NO: 139
CATATTAATGCGCAGGTA
SEQ ID NO: 140
ATATTAATGCGCAGGTAG
SEQ ID NO: 141
TATTAATGCGCAGGTAGC
SEQ ID NO: 142
ATTAATGCGCAGGTAGCA
SEQ ID NO: 143
TTAATGCGCAGGTAGCAA
SEQ ID NO: 144
TAATGCGCAGGTAGCAAA
SEQ ID NO: 145
GTGGTGTCACTCGTGACA
SEQ ID NO: 146
GTTTGCCTGGCACGATAT
SEQ ID NO: 147
CCTGGCACGATATTACGC
SEQ ID NO: 148
CTGGCACGATATTACGCA
SEQ ID NO: 149
TGGCACGATATTACGCAC
SEQ ID NO: 150
GGCACGATATTACGCACA
SEQ ID NO: 151
GCACGATATTACGCACAA
SEQ ID NO: 152
CACGATATTACGCACAAC
SEQ ID NO: 153
ACGATATTACGCACAACT
SEQ ID NO: 154
CGATATTACGCACAACTA
SEQ ID NO: 155
GAAAGTTTACGCCCTGAC
SEQ ID NO: 156
AAAGTTTACGCCCTGACA
SEQ ID NO: 157
AAGTTTACGCCCTGACAC
SEQ ID NO: 158
AGTTTACGCCCTGACACA
SEQ ID NO: 159
GTTTACGCCCTGACACAC
SEQ ID NO: 160
TTTACGCCCTGACACACG
SEQ ID NO: 161
TTACGCCCTGACACACGT
SEQ ID NO: 162
TACGCCCTGACACACGTT
SEQ ID NO: 163
ACGCCCTGACACACGTTA
SEQ ID NO: 164
CGCCCTGACACACGTTAT
SEQ ID NO: 165
GCCCTGACACACGTTATG
SEQ ID NO: 166
GAGTACTGTAGGCACGGC
SEQ ID NO: 167
GAAGCTGCGCTGTGCACC
SEQ ID NO: 168
ATCTAAAGTTGCGTAGTG
SEQ ID NO: 169
TCTAAAGTTGCGTAGTGA
SEQ ID NO: 170
CTATTACCTCTTACGCAA
SEQ ID NO: 171
TATTACCTCTTACGCAAT
SEQ ID NO: 172
ATTACCTCTTACGCAATA
SEQ ID NO: 173
TTACCTCTTACGCAATAT
SEQ ID NO: 174
TACCTCTTACGCAATATA
SEQ ID NO: 175
TCTTACGCAATATAATAG
SEQ ID NO: 176
TTCGTAAGTCTAATCATA
SEQ ID NO: 177
AGTTTGTTCGCATTCAAC
SEQ ID NO: 178
GGTGGTTTCTCAATCGAT
SEQ ID NO: 179
AAACTGGAATTGCCGTTT
SEQ ID NO: 180
AACTGGAATTGCCGTTTT
SEQ ID NO: 181
TATGCCTGCTAGTTGGGT
SEQ ID NO: 182
ATGCCTGCTAGTTGGGTG
SEQ ID NO: 183
GCACATCAGTAGTCTTAC
SEQ ID NO: 184
CACATCAGTAGTCTTACT
SEQ ID NO: 185
ACATCAGTAGTCTTACTC
SEQ ID NO: 186
CATCAGTAGTCTTACTCT
SEQ ID NO: 187
ATCAGTAGTCTTACTCTC
SEQ ID NO: 188
ATGAGCTTAGTCCTGTTG
SEQ ID NO: 189
TGAGCTTAGTCCTGTTGC
SEQ ID NO: 190
CTACGACAGATGTCTTGT
SEQ ID NO: 191
TGATGACAATGCGTTAGC
SEQ ID NO: 192
GATGACAATGCGTTAGCT
SEQ ID NO: 193
TGCGTTAGCTTACTACAA
SEQ ID NO: 194
CTAGCTAGTGGGGGACAA
SEQ ID NO: 195
TAGCTAGTGGGGGACAAC
SEQ ID NO: 196
AGCTAGTGGGGGACAACC
SEQ ID NO: 197
GCTAGTGGGGGACAACCA
SEQ ID NO: 198
CTAGTGGGGGACAACCAA
SEQ ID NO: 199
TAGTGGGGGACAACCAAT
SEQ ID NO: 200
CCGTTGCCACATAGATCA
SEQ ID NO: 201
CTGTACCGTCTGCGGTAT
SEQ ID NO: 202
TGTACCGTCTGCGGTATG
SEQ ID NO: 203
ACTCCGCGAACCCATGCT
SEQ ID NO: 204
CTCCGCGAACCCATGCTT
SEQ ID NO: 205
CAGCCCGTCTTACACCGT
SEQ ID NO: 206
AGCCCGTCTTACACCGTG
SEQ ID NO: 207
GCCCGTCTTACACCGTGC
SEQ ID NO: 208
CCCGTCTTACACCGTGCG
SEQ ID NO: 209
CCGTCTTACACCGTGCGG
SEQ ID NO: 210
CGTCTTACACCGTGCGGC
SEQ ID NO: 211
GTCTTACACCGTGCGGCA
SEQ ID NO: 212
TCTTACACCGTGCGGCAC
SEQ ID NO: 213
CTTACACCGTGCGGCACA
SEQ ID NO: 214
TTACACCGTGCGGCACAG
SEQ ID NO: 215
TACACCGTGCGGCACAGG
SEQ ID NO: 216
ACACCGTGCGGCACAGGC
SEQ ID NO: 217
CGGCACAGGCACTAGTAC
SEQ ID NO: 218
CGTATACGCCAACTTAGG
SEQ ID NO: 219
GTATACGCCAACTTAGGT
SEQ ID NO: 220
ACTTAGGTGAACGTGTAC
SEQ ID NO: 221
CTTAGGTGAACGTGTACG
SEQ ID NO: 222
GATGCCATGCGAAATGCT
SEQ ID NO: 223
ATGCCATGCGAAATGCTG
SEQ ID NO: 224
TGCCATGCGAAATGCTGG
SEQ ID NO: 225
GCCATGCGAAATGCTGGT
SEQ ID NO: 226
CTACAGTGTTCCCACCTA
SEQ ID NO: 227
TAGCTCTAGACTTAGTTT
SEQ ID NO: 228
ATCAGCGATTATGACTAC
SEQ ID NO: 229
TTATGACTACTATCGTTA
SEQ ID NO: 230
TATGACTACTATCGTTAT
SEQ ID NO: 231
ATGACTACTATCGTTATA
SEQ ID NO: 232
TTACGATGGTGGCTGTAT
SEQ ID NO: 233
GGTAAGGCTAGACTTTAT
SEQ ID NO: 234
AATAGCCGCCACTAGAGG
SEQ ID NO: 235
ATAGCCGCCACTAGAGGA
SEQ ID NO: 236
AACGTGTTGTAGCTTGTC
SEQ ID NO: 237
ACGTGTTGTAGCTTGTCA
SEQ ID NO: 238
TTGTAGCTTGTCACACCG
SEQ ID NO: 239
TGTAGCTTGTCACACCGT
SEQ ID NO: 240
GTAGCTTGTCACACCGTT
SEQ ID NO: 241
TAGCTTGTCACACCGTTT
SEQ ID NO: 242
TGGCGGTTCACTATATGT
SEQ ID NO: 243
GGCGGTTCACTATATGTT
SEQ ID NO: 244
GCGGTTCACTATATGTTA
SEQ ID NO: 245
AAAATTGCCGATAAGTAT
SEQ ID NO: 246
AAATTGCCGATAAGTATG
SEQ ID NO: 247
AATTGCCGATAAGTATGT
SEQ ID NO: 248
ATTGCCGATAAGTATGTC
SEQ ID NO: 249
TACGCATATTTGCGTAAA
SEQ ID NO: 250
ACGCATATTTGCGTAAAC
SEQ ID NO: 251
ATGATACTCTCTGACGAT
SEQ ID NO: 252
TGATACTCTCTGACGATG
SEQ ID NO: 253
GATACTCTCTGACGATGC
SEQ ID NO: 254
ATAGCACTTATGCATCTC
SEQ ID NO: 255
TAGCACTTATGCATCTCA
SEQ ID NO: 256
ATGATTGAACGGTTCGTG
SEQ ID NO: 257
TTGAACGGTTCGTGTCTT
SEQ ID NO: 258
GCATACGTAGACCATTCT
SEQ ID NO: 259
TGTTAATCCGTATGTTTG
SEQ ID NO: 260
GTTAATCCGTATGTTTGC
SEQ ID NO: 261
TTAATCCGTATGTTTGCA
SEQ ID NO: 262
GAGGTATGAGCTATTATT
SEQ ID NO: 263
ATGTGTTGGTAGCGATAA
SEQ ID NO: 264
TGTGTTGGTAGCGATAAT
SEQ ID NO: 265
GTGTTGGTAGCGATAATG
SEQ ID NO: 266
GCAGAAACGCTCAAAGCT
SEQ ID NO: 267
GGTATTGCTACTGTACGT
SEQ ID NO: 268
CTACTGTACGTGAAGTGC
SEQ ID NO: 269
GTACGTGAAGTGCTGTCT
SEQ ID NO: 270
CCACCACTTAACCGAAAT
SEQ ID NO: 271
CACCACTTAACCGAAATT
SEQ ID NO: 272
ACCACTTAACCGAAATTA
SEQ ID NO: 273
CCACTTAACCGAAATTAT
SEQ ID NO: 274
CACTTAACCGAAATTATG
SEQ ID NO: 275
ACTTAACCGAAATTATGT
SEQ ID NO: 276
CTTAACCGAAATTATGTC
SEQ ID NO: 277
TTAACCGAAATTATGTCT
SEQ ID NO: 278
GGTTATCGTGTAACTAAA
SEQ ID NO: 279
GTTGTTTACCGAGGTACA
SEQ ID NO: 280
TTGTTTACCGAGGTACAA
SEQ ID NO: 281
GTGCCACAAGAGCACTAT
SEQ ID NO: 282
GCTCTCATGCCGCTGTTG
SEQ ID NO: 283
CTCTCATGCCGCTGTTGA
SEQ ID NO: 284
TCTCATGCCGCTGTTGAT
SEQ ID NO: 285
CTCATGCCGCTGTTGATG
SEQ ID NO: 286
GCATTGCCTGAGACGACA
SEQ ID NO: 287
CATTGCCTGAGACGACAG
SEQ ID NO: 288
ATTGCCTGAGACGACAGC
SEQ ID NO: 289
ATGCCAGATTACGTGCTA
SEQ ID NO: 290
CAGATTACGTGCTAAGCA
SEQ ID NO: 291
TGTACATTGGCGACCCTG
SEQ ID NO: 292
TAAGGGTGTTATCACGCA
SEQ ID NO: 293
AAGGGTGTTATCACGCAT
SEQ ID NO: 294
GGCCACAAATAGGCGTGG
SEQ ID NO: 295
ATTCCTTACACGTAACCC
SEQ ID NO: 296
TTCCTTACACGTAACCCT
SEQ ID NO: 297
CGTAACCCTGCTTGGAGA
SEQ ID NO: 298
ATTCCACGTAGGAATGTG
SEQ ID NO: 299
TTCCACGTAGGAATGTGG
SEQ ID NO: 300
TCCACGTAGGAATGTGGC
SEQ ID NO: 301
CCACGTAGGAATGTGGCA
SEQ ID NO: 302
CACGTAGGAATGTGGCAA
SEQ ID NO: 303
ACGTAGGAATGTGGCAAC
SEQ ID NO: 304
CTCTATGATGGGTTTTAA
SEQ ID NO: 305
ACATGTACGTGCATGGAT
SEQ ID NO: 306
CATGTACGTGCATGGATT
SEQ ID NO: 307
CAGGTGTTAACCTAGTTG
SEQ ID NO: 308
AGGTGTTAACCTAGTTGC
SEQ ID NO: 309
GGAATGTAGTGCGTATAA
SEQ ID NO: 310
GAATGTAGTGCGTATAAA
SEQ ID NO: 311
AATGTAGTGCGTATAAAG
SEQ ID NO: 312
ATGTAGTGCGTATAAAGA
SEQ ID NO: 313
TGTAGTGCGTATAAAGAT
SEQ ID NO: 314
CTCTGACAGAGTCGTATT
SEQ ID NO: 315
TCTGACAGAGTCGTATTT
SEQ ID NO: 316
CTGACAGAGTCGTATTTG
SEQ ID NO: 317
GGACCTGAGCGCACCTGT
SEQ ID NO: 318
GACCTGAGCGCACCTGTT
SEQ ID NO: 319
ACCTGAGCGCACCTGTTG
SEQ ID NO: 320
TTACGTCTATAATCCGTT
SEQ ID NO: 321
TACGTCTATAATCCGTTT
SEQ ID NO: 322
ACGTCTATAATCCGTTTA
SEQ ID NO: 323
CGTCTATAATCCGTTTAT
SEQ ID NO: 324
GTCTATAATCCGTTTATG
SEQ ID NO: 325
TCTATAATCCGTTTATGA
SEQ ID NO: 326
TAATGCGGCTTGTAGAAA
SEQ ID NO: 327
AATGCGGCTTGTAGAAAG
SEQ ID NO: 328
ATGCGGCTTGTAGAAAGG
SEQ ID NO: 329
TGCGGCTTGTAGAAAGGT
SEQ ID NO: 330
ATTGCAATGTCGATAGAT
SEQ ID NO: 331
TTGCAATGTCGATAGATA
SEQ ID NO: 332
ACACTAGAGTGCTATCTA
SEQ ID NO: 333
CTGCTACGTGTATAACAC
SEQ ID NO: 334
GAGCTTTGGGCTAAGCGC
SEQ ID NO: 335
AGCTTTGGGCTAAGCGCA
SEQ ID NO: 336
GCTTTGGGCTAAGCGCAA
SEQ ID NO: 337
CTTTGGGCTAAGCGCAAC
SEQ ID NO: 338
TTTGGGCTAAGCGCAACA
SEQ ID NO: 339
TTGGGCTAAGCGCAACAT
SEQ ID NO: 340
TGGGCTAAGCGCAACATT
SEQ ID NO: 341
GGGCTAAGCGCAACATTA
SEQ ID NO: 342
GGCTAAGCGCAACATTAA
SEQ ID NO: 343
ATGGTCAAGTAGACTTAT
SEQ ID NO: 344
GAAATGCCCGTAATGGTG
SEQ ID NO: 345
CCTTCGAACATATCGTTT
SEQ ID NO: 346
CTTCGAACATATCGTTTA
SEQ ID NO: 347
TTCGAACATATCGTTTAT
SEQ ID NO: 348
TCGAACATATCGTTTATG
SEQ ID NO: 349
GTCATAGTCAGTTAGGTG
SEQ ID NO: 350
TCATAGTCAGTTAGGTGG
SEQ ID NO: 351
CATAGTCAGTTAGGTGGT
SEQ ID NO: 352
ATTGGACTAGCTAAACGT
SEQ ID NO: 353
TTGGACTAGCTAAACGTT
SEQ ID NO: 354
TACAATCTAGTCAAGCGT
SEQ ID NO: 355
ACAATCTAGTCAAGCGTG
SEQ ID NO: 356
CAATCTAGTCAAGCGTGG
SEQ ID NO: 357
AGCGTGGCAACCGGGTGT
SEQ ID NO: 358
GCGTGGCAACCGGGTGTT
SEQ ID NO: 359
GGAGTTGCACCAGGTACA
SEQ ID NO: 360
TACGACCCTAAGACTAAA
SEQ ID NO: 361
TGGGACACTTCGCATGGT
SEQ ID NO: 362
GGGACACTTCGCATGGTG
SEQ ID NO: 363
GGACACTTCGCATGGTGG
SEQ ID NO: 364
GACACTTCGCATGGTGGA
SEQ ID NO: 365
ACACTTCGCATGGTGGAC
SEQ ID NO: 366
ACTAATGTGAATGCGTCA
SEQ ID NO: 367
CTAATGTGAATGCGTCAT
SEQ ID NO: 368
TAATGTGAATGCGTCATC
SEQ ID NO: 369
AATGTGAATGCGTCATCA
SEQ ID NO: 370
ATCTTGGCAAACCACGCG
SEQ ID NO: 371
TCTTGGCAAACCACGCGA
SEQ ID NO: 372
CTTGGCAAACCACGCGAA
SEQ ID NO: 373
CAAACCACGCGAACAAAT
SEQ ID NO: 374
AAACCACGCGAACAAATA
SEQ ID NO: 375
AACCACGCGAACAAATAG
SEQ ID NO: 376
ACCACGCGAACAAATAGA
SEQ ID NO: 377
TTGTTAATAACGCTACTA
SEQ ID NO: 378
TATTCTAAGCACACGCCT
SEQ ID NO: 379
ATTCTAAGCACACGCCTA
SEQ ID NO: 380
TTAATTTAGTGCGTGATC
SEQ ID NO: 381
TAATTTAGTGCGTGATCT
SEQ ID NO: 382
AATTTAGTGCGTGATCTC
SEQ ID NO: 383
ATTTAGTGCGTGATCTCC
SEQ ID NO: 384
TTTAGTGCGTGATCTCCC
SEQ ID NO: 385
AAAGTGTACGTTGAAATC
SEQ ID NO: 386
ATCAGGCCGGTAGCACAC
SEQ ID NO: 387
TCAGGCCGGTAGCACACC
SEQ ID NO: 388
CAGGCCGGTAGCACACCT
SEQ ID NO: 389
AGGCCGGTAGCACACCTT
SEQ ID NO: 390
GGCCGGTAGCACACCTTG
SEQ ID NO: 391
GCCGGTAGCACACCTTGT
SEQ ID NO: 392
GTTCTTACTGAGTCTAAC
SEQ ID NO: 393
TTCTTACTGAGTCTAACA
SEQ ID NO: 394
GGTGCAGGTATATGCGCT
SEQ ID NO: 395
GTGCAGGTATATGCGCTA
SEQ ID NO: 396
TGCAGGTATATGCGCTAG
SEQ ID NO: 397
GCGCTAGTTATCAGACTC
SEQ ID NO: 398
TTAAACCGTGCTTTAACT
SEQ ID NO: 399
CACAAAAGTTTAACGGCC
SEQ ID NO: 400
ACAAAAGTTTAACGGCCT
SEQ ID NO: 401
CAAAAGTTTAACGGCCTT
SEQ ID NO: 402
AAAAGTTTAACGGCCTTA
SEQ ID NO: 403
AAAGTTTAACGGCCTTAC
SEQ ID NO: 404
AAGTTTAACGGCCTTACT
SEQ ID NO: 405
AGTTTAACGGCCTTACTG
SEQ ID NO: 406
GTTTAACGGCCTTACTGT
SEQ ID NO: 407
TTTAACGGCCTTACTGTT
SEQ ID NO: 408
TGGTTGGACCTTTGGTGC
SEQ ID NO: 409
ACTTTCCTCGTGAAGGTG
SEQ ID NO: 410
AAATTGACCGCCTCAATG
SEQ ID NO: 411
AATTGACCGCCTCAATGA
SEQ ID NO: 412
ATTGACCGCCTCAATGAG
SEQ ID NO: 413
TTGACCGCCTCAATGAGG
SEQ ID NO: 414
TGACCGCCTCAATGAGGT
SEQ ID NO: 415
GACCGCCTCAATGAGGTT
SEQ ID NO: 416
ACCGCCTCAATGAGGTTG
SEQ ID NO: 417
AGACGACTCTGAGCCAGT
SEQ ID NO: 418
ATTTTGTTCGCGCTACTG
SEQ ID NO: 419
TTTTGTTCGCGCTACTGC
SEQ ID NO: 420
GTTCGCGCTACTGCAACG
SEQ ID NO: 421
TTCGCGCTACTGCAACGA
SEQ ID NO: 422
TCGCGCTACTGCAACGAT
SEQ ID NO: 423
TTTCGGATGGCTTATTGT
SEQ ID NO: 424
TTCGGATGGCTTATTGTT
SEQ ID NO: 425
TCGGATGGCTTATTGTTG
SEQ ID NO: 426
ATGGCTTATTGTTGGCGT
SEQ ID NO: 427
TGGCTTATTGTTGGCGTT
SEQ ID NO: 428
TCACACAATCGACGGTTC
SEQ ID NO: 429
CACACAATCGACGGTTCA
SEQ ID NO: 430
ACACAATCGACGGTTCAT
SEQ ID NO: 431
CACAATCGACGGTTCATC
SEQ ID NO: 432
TATGATGAACCGACGACG
SEQ ID NO: 433
ATGATGAACCGACGACGA
SEQ ID NO: 434
ACAAGCTGATGAGTACGA
SEQ ID NO: 435
CAAGCTGATGAGTACGAA
SEQ ID NO: 436
AAGCTGATGAGTACGAAC
SEQ ID NO: 437
AGCTGATGAGTACGAACT
SEQ ID NO: 438
GCTGATGAGTACGAACTT
SEQ ID NO: 439
CTGATGAGTACGAACTTA
SEQ ID NO: 440
TGATGAGTACGAACTTAT
SEQ ID NO: 441
GATGAGTACGAACTTATG
SEQ ID NO: 442
GAGACAGGTACGTTAATA
SEQ ID NO: 443
AGACAGGTACGTTAATAG
SEQ ID NO: 444
GACAGGTACGTTAATAGT
SEQ ID NO: 445
AATAGCGTACTTCTTTTT
SEQ ID NO: 446
GCGCTTCGATTGTGTGCG
SEQ ID NO: 447
CGCTTCGATTGTGTGCGT
SEQ ID NO: 448
GCTTCGATTGTGTGCGTA
SEQ ID NO: 449
CTTCGATTGTGTGCGTAC
SEQ ID NO: 450
TTCGATTGTGTGCGTACT
SEQ ID NO: 451
TTTACGTTTACTCTCGTG
SEQ ID NO: 452
TTACGTTTACTCTCGTGT
SEQ ID NO: 453
GGCAGATTCCAACGGTAC
SEQ ID NO: 454
GCAGATTCCAACGGTACT
SEQ ID NO: 455
CAGATTCCAACGGTACTA
SEQ ID NO: 456
AGATTCCAACGGTACTAT
SEQ ID NO: 457
GATTCCAACGGTACTATT
SEQ ID NO: 458
CTATTACCGTTGAAGAGC
SEQ ID NO: 459
TATTACCGTTGAAGAGCT
SEQ ID NO: 460
GCGCGTACGCGTTCCATG
SEQ ID NO: 461
CGCGTACGCGTTCCATGT
SEQ ID NO: 462
TATTCTGACCAGACCGCT
SEQ ID NO: 463
ATTCTGACCAGACCGCTT
SEQ ID NO: 464
AAGTGAACTCGTAATCGG
SEQ ID NO: 465
AGTGAACTCGTAATCGGA
SEQ ID NO: 466
GTGAACTCGTAATCGGAG
SEQ ID NO: 467
TGAACTCGTAATCGGAGC
SEQ ID NO: 468
GAACTCGTAATCGGAGCT
SEQ ID NO: 469
AACTCGTAATCGGAGCTG
SEQ ID NO: 470
ACTCGTAATCGGAGCTGT
SEQ ID NO: 471
CTCGTAATCGGAGCTGTG
SEQ ID NO: 472
TCGTAATCGGAGCTGTGA
SEQ ID NO: 473
CGTAATCGGAGCTGTGAT
SEQ ID NO: 474
GTAATCGGAGCTGTGATC
SEQ ID NO: 475
TAATCGGAGCTGTGATCC
SEQ ID NO: 476
CTGTTGCTACATCACGAA
SEQ ID NO: 477
TGTTGCTACATCACGAAC
SEQ ID NO: 478
GTTGCTACATCACGAACG
SEQ ID NO: 479
TTGCTACATCACGAACGC
SEQ ID NO: 480
TGCTACATCACGAACGCT
SEQ ID NO: 481
GCTACATCACGAACGCTT
SEQ ID NO: 482
CTACATCACGAACGCTTT
SEQ ID NO: 483
CAAATTGGGAGCTTCGCA
SEQ ID NO: 484
TTGCTGCATACAGTCGCT
SEQ ID NO: 485
TGCTGCATACAGTCGCTA
SEQ ID NO: 486
GCTGCATACAGTCGCTAC
SEQ ID NO: 487
CTGCATACAGTCGCTACA
SEQ ID NO: 488
TGCATACAGTCGCTACAG
SEQ ID NO: 489
GGAGATTGATTAAACGAA
SEQ ID NO: 490
GATAACACTCGCTACTTG
SEQ ID NO: 491
ATAACACTCGCTACTTGT
SEQ ID NO: 492
TAACACTCGCTACTTGTG
SEQ ID NO: 493
AACACTCGCTACTTGTGA
SEQ ID NO: 494
ACACTCGCTACTTGTGAG
SEQ ID NO: 495
CACTCGCTACTTGTGAGC
SEQ ID NO: 496
ACTCGCTACTTGTGAGCT
SEQ ID NO: 497
CTCGCTACTTGTGAGCTT
SEQ ID NO: 498
TACGAGGGCAATTCACCA
SEQ ID NO: 499
TCCTGACGGCGTAAAACA
SEQ ID NO: 500
CCTGACGGCGTAAAACAC
SEQ ID NO: 501
CTGACGGCGTAAAACACG
SEQ ID NO: 502
TGACGGCGTAAAACACGT
SEQ ID NO: 503
GTCTATCAGTTACGTGCC
SEQ ID NO: 504
TCTATCAGTTACGTGCCA
SEQ ID NO: 505
CTATCAGTTACGTGCCAG
SEQ ID NO: 506
ATATCGGTAATTATACAG
SEQ ID NO: 507
TATCGGTAATTATACAGT
SEQ ID NO: 508
ATCGGTAATTATACAGTT
SEQ ID NO: 509
TTAGAGTATCATGACGTT
SEQ ID NO: 510
TAGAGTATCATGACGTTC
SEQ ID NO: 511
AGAGTATCATGACGTTCG
SEQ ID NO: 512
GAGTATCATGACGTTCGT
SEQ ID NO: 513
CATTACGTTTGGTGGACC
SEQ ID NO: 514
GTCTTGGTTCACCGCTCT
SEQ ID NO: 515
TCTTGGTTCACCGCTCTC
SEQ ID NO: 516
CACCGCTCTCACTCAACA
SEQ ID NO: 517
ACCGCTCTCACTCAACAT
SEQ ID NO: 518
CAAATTGGCTACTACCGA
SEQ ID NO: 519
AAATTGGCTACTACCGAA
SEQ ID NO: 520
AATTGGCTACTACCGAAG
SEQ ID NO: 521
ATTGGCTACTACCGAAGA
SEQ ID NO: 522
TTGGCTACTACCGAAGAG
SEQ ID NO: 523
TGGCTACTACCGAAGAGC
SEQ ID NO: 524
TACTACCGAAGAGCTACC
SEQ ID NO: 525
TCATATGGGTTGCAACTG
SEQ ID NO: 526
CATATGGGTTGCAACTGA
SEQ ID NO: 527
TCACATTGGCACCCGCAA
SEQ ID NO: 528
CACATTGGCACCCGCAAT
SEQ ID NO: 529
GGCGGCAGTCAAGCCTCT
SEQ ID NO: 530
GCGGCAGTCAAGCCTCTT
SEQ ID NO: 531
CACGTAGTCGCAACAGTT
SEQ ID NO: 532
ACGTAGTCGCAACAGTTC
SEQ ID NO: 533
GCTTCAGCGTTCTTCGGA
SEQ ID NO: 534
CTTCAGCGTTCTTCGGAA
SEQ ID NO: 535
TTCAGCGTTCTTCGGAAT
SEQ ID NO: 536
TCAGCGTTCTTCGGAATG
SEQ ID NO: 537
GCCTTACCGCAGAGACAG
SEQ ID NO: 538
TTTCGCTTTTCCGTTTAC
SEQ ID NO: 539
TTCGCTTTTCCGTTTACG
SEQ ID NO: 540
TCGCTTTTCCGTTTACGA
SEQ ID NO: 541
CGCTTTTCCGTTTACGAT
SEQ ID NO: 542
GCTTTTCCGTTTACGATA
SEQ ID NO: 543
CTTTTCCGTTTACGATAT
SEQ ID NO: 544
GAATGAATTCTCGTAACT
SEQ ID NO: 545
AATGAATTCTCGTAACTA
SEQ ID NO: 546
ATGAATTCTCGTAACTAC
SEQ ID NO: 547
TGAATTCTCGTAACTACA
SEQ ID NO: 548
GAATTCTCGTAACTACAT
SEQ ID NO: 549
AATTCTCGTAACTACATA
SEQ ID NO: 550
ATTCTCGTAACTACATAG
SEQ ID NO: 551
TTCTCGTAACTACATAGC
SEQ ID NO: 552
TCTCGTAACTACATAGCA
SEQ ID NO: 553
CTCGTAACTACATAGCAC
SEQ ID NO: 554
GTACGATCGAGTGTACAG
SEQ ID NO: 555
TACGATCGAGTGTACAGT
SEQ ID NO: 556
ACGATCGAGTGTACAGTG
SEQ ID NO: 557
CGATCGAGTGTACAGTGA
SEQ ID NO: 558
GATCGAGTGTACAGTGAA
SEQ ID NO: 559
ATCGAGTGTACAGTGAAC

### EXAMPLE

What follows describes a method for identifying primers that are:
specific to the genome of a virus V₀ that infects a given host H;
is absent from the genomes of all other viruses V_{Host}-V₀ that infect host H;
is absent from the genomes of select bacterial or fungal pathogens P_{select} that infect host H;
is absent from the full-length genome of host H; and,
is absent from the spliced protein-coding or non-protein-coding transcripts that are known for host H.
   These primers can be used to determine the presence of V₀ in a biological sample (tissue or bodily fluid).
   The incarnation of the method is described below for the specific case where we made the following selections:
V₀ is SARS-CoV-2 (NC 045512)
H is Homo sapiens
V_{Host} is a collection of the genomes of 400 viruses that infect humans (downloaded from the National Center for Biotechnology Information on Apr. 1, 2020)/V_{Host} includes V₀
P_{select} is a collection comprising the genomes of Pseudomonas aeruginosa PAO1 (NC_002516.2), Neisseria meningitis MC58 (NC_003112.2), Staphylococcus aureus subsp. aureus NCTC 8325 (NC_007795.1), and Candida albicans SC5314 (NC_032089.1)
the genome assembly used for Homo sapiens is the one denoted GRCh38
the spliced protein-coding transcripts of Homo sapiens are those that are described in ENSEMBL Rel. 96
the non-protein-coding transcripts of Homo sapiens are those that are described in ENSEMBL Rel. 96
   Clearly, additional non-viral pathogens can be included in P_{select}. Also, other viral genomes can be included in V_{Host} as they become available. Also, V₀ may include more than one strain.
   Results: the candidate sequence segments that can be used to compose the pairs of primers to use to detect SARS-CoV-2 are listed in the accompanying Excel file.

### Method

Remove the genome of V₀ from the collection of genomes V_{Host} to give rise to {V_{Host}−V₀}.
   Note: In the preferred incarnation, the set {V_{Host}−V₀} comprises all the viruses that infect humans except for SARS-CoV-2.
Enumerate all possible k-mers (groups of k consecutive letters) that can be found in the forward and reverse strands of all genomes in the collection {V_{Host}−V₀}. A preferred choice for k is 12. However, other choices of k, smaller or bigger than 12 can be used. By modifying the value of k the sensitivity and the specificity of the method can be adjusted. Let this set of k-mers be called K.
   Note: In the preferred incarnation, K is the set of all 12-mers that can be found in sense or antisense orientation in the genomes of all known human viruses other than SARS-CoV-2.
Enumerate all possible m₁-mers (groups of m₁ consecutive letters) that can be found in the forward strand only of the genome of V₀. A preferred choice for m₁ is 18. Also, m₁≥k. However, other choices of m₁, smaller or bigger than 18 can be made. By modifying the value of m₁, the sensitivity and specificity of the method can be adjusted. Let this set of m₁-mers be called M_{candidates}. The set M_{candidates} contains a set of candidate sequence segments from which to form pairs of primers that can be used to diagnose V₀.
   Note: In the preferred incarnation, M_{candidates} is the set of all 18-mers that can be found in sense orientation in the genome of SARS-CoV-2.
Enumerate all possible m₂-mers (groups of m₂ consecutive letters) that can be found in the forward strand of the genome of V₀. A preferred choice for m₂ is 16. Also, m₂≥k and m₂<m₁. Other choices of m₂, smaller or bigger than 16 can be made. By modifying the value of m₂ the sensitivity and specificity of the method can be adjusted. Let this set of m₂-mers be called Mₘ₂.
   Note: In the preferred incarnation, Mₘ₂ is the set of all 16-mers that can be found in sense orientation in the genome of V₀.
Identify the subset of all m₂-mers of Mₘ₂ that can be found in sense or antisense orientation in the genome of host H: let this subset of m₂-mers be called M_{Host}.
   Note: In the preferred incarnation, M_{Host} is the set of all 16-mers that are found in sense orientation in the genome of SARS-CoV-2 as well as in sense or antisense orientation in the human genome.
Identify the subset of all m₂-mers of Mₘ₂ that can be found in sense or antisense orientation in the genomes of the pathogens in collection P_{select}: let this subset of m₂-mers be called M_{p-select}.
   Note: In the preferred incarnation, M_{p-select} is the set of all 16-mers that are found in sense orientation in the genome of SARS-CoV-2 as well as in sense or antisense orientation in the genomes of the four non-viral human pathogens listed above.
Identify the subset of all m₂-mers of Mₘ₂ that can be found in sense or antisense orientation in the spliced protein-coding and non-protein coding transcripts of the host genome H: let this set of m₂-mers be called M_{spliced}.
   Note: In the preferred incarnation, M_{spliced} is the set of all 16-mers that are found in sense orientation in the genome of SARS-CoV-2 as well as in sense or antisense orientation in the genomes of the spliced protein-coding and non protein coding transcripts of Homo sapiens.
Form the union of Mᵤₙᵢₒₙ=M_{Host} U M_{p-select} U M_{spliced}
   Note: In the preferred incarnation, Mᵤₙᵢₒₙ is the set of all 16-mers that are found in sense orientation in the genome of SARS-CoV-2 as well as in sense or antisense orientation in either the human genome, or the genomes of other human pathogens, or the spliced protein-coding and non-protein coding human transcripts.
Remove from M_{candidates} all of the m₁-mers in it that contain
   one or more m₂-mers of Mᵤₙᵢₒₙ as a substring; or,
   one or more k-mers from K as a substring.
Call the resulting set M_{final}. Use the m₁-mers contained in M_{final} to generate pairs of primers to diagnose the presence of V₀ in a sample.
   Note: In the preferred incarnation, M_{final} is the set of all 18-mers that are found in sense orientation in the genome of SARS-CoV-2 and
   do not contain a 16-mer substring that can be found in sense or antisense orientation in the human genome, in the genomes of other human pathogens, or in the spliced protein-coding and non-protein coding human transcripts and
   do not contain a 12-mer substring that can be found in sense or antisense orientation in any of the non-SARS-CoV-2 viral genomes that affect humans.
Optionally, further filter the set M_{final} to keep only those m₁-mers that satisfy one or more of the following properties:
   have melting temperature within a specific desired range
   have sequence composition within a specific desired range
   have specific nucleotides at their 5′ or 3′ termini
   are located on the genome at locations whose distances satisfy a minimum distance and a maximum distance constraint
   target a specific region of the genome of V₀
   target a specific protein-coding or non-protein coding transcript of V₀
Optionally, the non-viral human pathogens could be combined and processed with the human viruses of {V_{Host}−V₀}. In that case, the set M_{p-select} mentioned above will be the empty set.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of detecting SARS-CoV-2 in a sample obtained from a subject, the method comprising:
contacting the sample with a composition comprising a plurality of PCR primers targeting at least one sequence selected from the group consisting of SEQ ID NOS: 1-559, thereby forming a PCR mixture;
conducting PCR amplification of the PCR mixture and detection of the at least one sequence selected from the group consisting of SEQ ID NOS: 1-559;
wherein an elevated level of the at least one sequence selected from the group consisting SEQ ID NOS: 1-559 indicates the presence of SARS-CoV-2 in the sample.

2. The method according to claim 1, wherein the sample is a blood sample or a saliva sample.


SEQUENCE LISTING (560 sequences)

SEQ ID NO: 1 - DNA, 18 bp, Artificial Sequence
  Chemically synthesized
ttataccttcccaggtaa

SEQ ID NO: 2 - DNA, 18 bp, Artificial Sequence
  Chemically synthesized
gtcactcggctgcatgct

SEQ ID NO: 3 - DNA, 18 bp, Artificial Sequence
  Chemically synthesized
gcttagtgcactcacgca

SEQ ID NO: 4 - DNA, 18 bp, Artificial Sequence
  Chemically synthesized
cttagtgcactcacgcag

SEQ ID NO: 5 - DNA, 18 bp, Artificial Sequence
  Chemically synthesized
aggctgcttacggtttcg

SEQ ID NO: 6 - DNA, 18 bp, Artificial Sequence
  Chemically synthesized
ggctgcttacggtttcgt

SEQ ID NO: 7 - DNA, 18 bp, Artificial Sequence
  Chemically synthesized
ttttacaggttcgcgacg

SEQ ID NO: 8 - DNA, 18 bp, Artificial Sequence
  Chemically synthesized
tttacaggttcgcgacgt

SEQ ID NO: 9 - DNA, 18 bp, Artificial Sequence
  Chemically synthesized
ttacaggttcgcgacgtg

SEQ ID NO: 10 - DNA, 18 bp, Artificial Sequence
  Chemically synthesized
tacaggttcgcgacgtgc

SEQ ID NO: 11 - DNA, 18 bp, Artificial Sequence
  Chemically synthesized
acaggttcgcgacgtgct

SEQ ID NO: 12 - DNA, 18 bp, Artificial Sequence
  Chemically synthesized
ggtcttatcagaggcacg

SEQ ID NO: 13 - DNA, 18 bp, Artificial Sequence
  Chemically synthesized
gtcttatcagaggcacgt

SEQ ID NO: 14 - DNA, 18 bp, Artificial Sequence
  Chemically synthesized
catcaaacgttcggatgc

SEQ ID NO: 15 - DNA, 18 bp, Artificial Sequence
  Chemically synthesized
atcaaacgttcggatgct

SEQ ID NO: 16 - DNA, 18 bp, Artificial Sequence
  Chemically synthesized
tcaaacgttcggatgctc

SEQ ID NO: 17 - DNA, 18 bp, Artificial Sequence
  Chemically synthesized
caaacgttcggatgctcg

SEQ ID NO: 18 - DNA, 18 bp, Artificial Sequence
  Chemically synthesized
aaacgttcggatgctcga

SEQ ID NO: 19 - DNA, 18 bp, Artificial Sequence
  Chemically synthesized
aacgttcggatgctcgaa

SEQ ID NO: 20 - DNA, 18 bp, Artificial Sequence
  Chemically synthesized
acgttcggatgctcgaac

SEQ ID NO: 21 - DNA, 18 bp, Artificial Sequence
  Chemically synthesized
cgttcggatgctcgaact

SEQ ID NO: 22 - DNA, 18 bp, Artificial Sequence
  Chemically synthesized
gttcggatgctcgaactg

SEQ ID NO: 23 - DNA, 18 bp, Artificial Sequence
  Chemically synthesized
tggtagcagaactcgaag

SEQ ID NO: 24 - DNA, 18 bp, Artificial Sequence
  Chemically synthesized
ggtagcagaactcgaagg

SEQ ID NO: 25 - DNA, 18 bp, Artificial Sequence
  Chemically synthesized
aactcgaaggcattcagt

SEQ ID NO: 26 - DNA, 18 bp, Artificial Sequence
  Chemically synthesized
actcgaaggcattcagta

SEQ ID NO: 27 - DNA, 18 bp, Artificial Sequence
  Chemically synthesized
ctcgaaggcattcagtac

SEQ ID NO: 28 - DNA, 18 bp, Artificial Sequence
  Chemically synthesized
tcgaaggcattcagtacg

SEQ ID NO: 29 - DNA, 18 bp, Artificial Sequence
  Chemically synthesized
cgaaggcattcagtacgg

SEQ ID NO: 30 - DNA, 18 bp, Artificial Sequence
  Chemically synthesized
gaaggcattcagtacggt

SEQ ID NO: 31 - DNA, 18 bp, Artificial Sequence
  Chemically synthesized
aaggcattcagtacggtc

SEQ ID NO: 32 - DNA, 18 bp, Artificial Sequence
  Chemically synthesized
aggcattcagtacggtcg

SEQ ID NO: 33 - DNA, 18 bp, Artificial Sequence
  Chemically synthesized
ggcattcagtacggtcgt

SEQ ID NO: 34 - DNA, 18 bp, Artificial Sequence
  Chemically synthesized
gcattcagtacggtcgta

SEQ ID NO: 35 - DNA, 18 bp, Artificial Sequence
  Chemically synthesized
cattcagtacggtcgtag

SEQ ID NO: 36 - DNA, 18 bp, Artificial Sequence
  Chemically synthesized
ggtcgtagtggtgagaca

SEQ ID NO: 37 - DNA, 18 bp, Artificial Sequence
  Chemically synthesized
ccctcatgtgggcgaaat

SEQ ID NO: 38 - DNA, 18 bp, Artificial Sequence
  Chemically synthesized
cctcatgtgggcgaaata

SEQ ID NO: 39 - DNA, 18 bp, Artificial Sequence
  Chemically synthesized
ctcatgtgggcgaaatac

SEQ ID NO: 40 - DNA, 18 bp, Artificial Sequence
  Chemically synthesized
tcatgtgggcgaaatacc

SEQ ID NO: 41 - DNA, 18 bp, Artificial Sequence
  Chemically synthesized
catgtgggcgaaatacca

SEQ ID NO: 42 - DNA, 18 bp, Artificial Sequence
  Chemically synthesized
tgggcgaaataccagtgg

SEQ ID NO: 43 - DNA, 18 bp, Artificial Sequence
  Chemically synthesized
gggcgaaataccagtggc

SEQ ID NO: 44 - DNA, 18 bp, Artificial Sequence
  Chemically synthesized
cagtggcttaccgcaagg

SEQ ID NO: 45 - DNA, 18 bp, Artificial Sequence
  Chemically synthesized
agtggcttaccgcaaggt

SEQ ID NO: 46 - DNA, 18 bp, Artificial Sequence
  Chemically synthesized
gtggcttaccgcaaggtt

SEQ ID NO: 47 - DNA, 18 bp, Artificial Sequence
  Chemically synthesized
tggcttaccgcaaggttc

SEQ ID NO: 48 - DNA, 18 bp, Artificial Sequence
  Chemically synthesized
ggcttaccgcaaggttct

SEQ ID NO: 49 - DNA, 18 bp, Artificial Sequence
  Chemically synthesized
gcttaccgcaaggttctt

SEQ ID NO: 50 - DNA, 18 bp, Artificial Sequence
  Chemically synthesized
cttaccgcaaggttcttc

SEQ ID NO: 51 - DNA, 18 bp, Artificial Sequence
  Chemically synthesized
ttaccgcaaggttcttct

SEQ ID NO: 52 - DNA, 18 bp, Artificial Sequence
  Chemically synthesized
cttcttcgtaagaacggt

SEQ ID NO: 53 - DNA, 18 bp, Artificial Sequence
  Chemically synthesized
catgcgtgagcttaacgg

SEQ ID NO: 54 - DNA, 18 bp, Artificial Sequence
  Chemically synthesized
atgcgtgagcttaacgga

SEQ ID NO: 55 - DNA, 18 bp, Artificial Sequence
  Chemically synthesized
tgcgtgagcttaacggag

SEQ ID NO: 56 - DNA, 18 bp, Artificial Sequence
  Chemically synthesized
gcgtgagcttaacggagg

SEQ ID NO: 57 - DNA, 18 bp, Artificial Sequence
  Chemically synthesized
cgtgagcttaacggaggg

SEQ ID NO: 58 - DNA, 18 bp, Artificial Sequence
  Chemically synthesized
gtgagcttaacggagggg

SEQ ID NO: 59 - DNA, 18 bp, Artificial Sequence
  Chemically synthesized
gggcatacactcgctatg

SEQ ID NO: 60 - DNA, 18 bp, Artificial Sequence
  Chemically synthesized
ggcatacactcgctatgt

SEQ ID NO: 61 - DNA, 18 bp, Artificial Sequence
  Chemically synthesized
gcatacactcgctatgtc

SEQ ID NO: 62 - DNA, 18 bp, Artificial Sequence
  Chemically synthesized
catacactcgctatgtcg

SEQ ID NO: 63 - DNA, 18 bp, Artificial Sequence
  Chemically synthesized
atacactcgctatgtcga

SEQ ID NO: 64 - DNA, 18 bp, Artificial Sequence
  Chemically synthesized
tacactcgctatgtcgat

SEQ ID NO: 65 - DNA, 18 bp, Artificial Sequence
  Chemically synthesized
acactcgctatgtcgata

SEQ ID NO: 66 - DNA, 18 bp, Artificial Sequence
  Chemically synthesized
cactcgctatgtcgataa

SEQ ID NO: 67 - DNA, 18 bp, Artificial Sequence
  Chemically synthesized
tggctaccctcttgagtg

SEQ ID NO: 68 - DNA, 18 bp, Artificial Sequence
  Chemically synthesized
atgggtagaattcgatct

SEQ ID NO: 69 - DNA, 18 bp, Artificial Sequence
  Chemically synthesized
cagttgcgtcaccaaatg

SEQ ID NO: 70 - DNA, 18 bp, Artificial Sequence
  Chemically synthesized
tgagcatagtcttgccga

SEQ ID NO: 71 - DNA, 18 bp, Artificial Sequence
  Chemically synthesized
gagcatagtcttgccgaa

SEQ ID NO: 72 - DNA, 18 bp, Artificial Sequence
  Chemically synthesized
agcatagtcttgccgaat

SEQ ID NO: 73 - DNA, 18 bp, Artificial Sequence
  Chemically synthesized
gcatagtcttgccgaata

SEQ ID NO: 74 - DNA, 18 bp, Artificial Sequence
  Chemically synthesized
catagtcttgccgaatac

SEQ ID NO: 75 - DNA, 18 bp, Artificial Sequence
  Chemically synthesized
atagtcttgccgaatacc

SEQ ID NO: 76 - DNA, 18 bp, Artificial Sequence
  Chemically synthesized
tagtcttgccgaatacca

SEQ ID NO: 77 - DNA, 18 bp, Artificial Sequence
  Chemically synthesized
agtcttgccgaataccat

SEQ ID NO: 78 - DNA, 18 bp, Artificial Sequence
  Chemically synthesized
gtcttgccgaataccata

SEQ ID NO: 79 - DNA, 18 bp, Artificial Sequence
  Chemically synthesized
tcttgccgaataccataa

SEQ ID NO: 80 - DNA, 18 bp, Artificial Sequence
  Chemically synthesized
attcttcgtaagggtggt

SEQ ID NO: 81 - DNA, 18 bp, Artificial Sequence
  Chemically synthesized
ttcttcgtaagggtggtc

SEQ ID NO: 82 - DNA, 18 bp, Artificial Sequence
  Chemically synthesized
ttccacgtgctagcgcta

SEQ ID NO: 83 - DNA, 18 bp, Artificial Sequence
  Chemically synthesized
ggctgctcgtgttgtacg

SEQ ID NO: 84 - DNA, 18 bp, Artificial Sequence
  Chemically synthesized
gctgctcgtgttgtacga

SEQ ID NO: 85 - DNA, 18 bp, Artificial Sequence
  Chemically synthesized
ctgctcgtgttgtacgat

SEQ ID NO: 86 - DNA, 18 bp, Artificial Sequence
  Chemically synthesized
tgctcgtgttgtacgatc

SEQ ID NO: 87 - DNA, 18 bp, Artificial Sequence
  Chemically synthesized
gctcgtgttgtacgatca

SEQ ID NO: 88 - DNA, 18 bp, Artificial Sequence
  Chemically synthesized
ctcgtgttgtacgatcaa

SEQ ID NO: 89 - DNA, 18 bp, Artificial Sequence
  Chemically synthesized
tcgtgttgtacgatcaat

SEQ ID NO: 90 - DNA, 18 bp, Artificial Sequence
  Chemically synthesized
cgtgttgtacgatcaatt

SEQ ID NO: 91 - DNA, 18 bp, Artificial Sequence
  Chemically synthesized
gtgttgtacgatcaattt

SEQ ID NO: 92 - DNA, 18 bp, Artificial Sequence
  Chemically synthesized
tgttgtacgatcaatttt

SEQ ID NO: 93 - DNA, 18 bp, Artificial Sequence
  Chemically synthesized
gttgtacgatcaattttc

SEQ ID NO: 94 - DNA, 18 bp, Artificial Sequence
  Chemically synthesized
ttgtacgatcaattttct

SEQ ID NO: 95 - DNA, 18 bp, Artificial Sequence
  Chemically synthesized
ttgacttcgcagtggcta

SEQ ID NO: 96 - DNA, 18 bp, Artificial Sequence
  Chemically synthesized
tgacttcgcagtggctaa

SEQ ID NO: 97 - DNA, 18 bp, Artificial Sequence
  Chemically synthesized
gacttcgcagtggctaac

SEQ ID NO: 98 - DNA, 18 bp, Artificial Sequence
  Chemically synthesized
acttcgcagtggctaact

SEQ ID NO: 99 - DNA, 18 bp, Artificial Sequence
  Chemically synthesized
cttcgcagtggctaacta

SEQ ID NO: 100 - DNA, 18 bp, Artificial Sequence
  Chemically synthesized
aaactcaaacccgtcctt

SEQ ID NO: 101 - DNA, 18 bp, Artificial Sequence
  Chemically synthesized
aactcaaacccgtccttg

SEQ ID NO: 102 - DNA, 18 bp, Artificial Sequence
  Chemically synthesized
tcttagagacggttggga

SEQ ID NO: 103 - DNA, 18 bp, Artificial Sequence
  Chemically synthesized
cttagagacggttgggaa

SEQ ID NO: 104 - DNA, 18 bp, Artificial Sequence
  Chemically synthesized
ttagagacggttgggaaa

SEQ ID NO: 105 - DNA, 18 bp, Artificial Sequence
  Chemically synthesized
gtatggctacatactact

SEQ ID NO: 106 - DNA, 18 bp, Artificial Sequence
  Chemically synthesized
tttaagcggacacaatct

SEQ ID NO: 107 - DNA, 18 bp, Artificial Sequence
  Chemically synthesized
ttaagcggacacaatctt

SEQ ID NO: 108 - DNA, 18 bp, Artificial Sequence
  Chemically synthesized
tgttgtcggcccaaatgt

SEQ ID NO: 109 - DNA, 18 bp, Artificial Sequence
  Chemically synthesized
acttacccgggtcagggt

SEQ ID NO: 110 - DNA, 18 bp, Artificial Sequence
  Chemically synthesized
gaatttgcgagaaatgct

SEQ ID NO: 111 - DNA, 18 bp, Artificial Sequence
  Chemically synthesized
actgtagcgtcacttatc

SEQ ID NO: 112 - DNA, 18 bp, Artificial Sequence
  Chemically synthesized
ctgtagcgtcacttatca

SEQ ID NO: 113 - DNA, 18 bp, Artificial Sequence
  Chemically synthesized
acacacttaacgatctaa

SEQ ID NO: 114 - DNA, 18 bp, Artificial Sequence
  Chemically synthesized
gctgttacagcgtataat

SEQ ID NO: 115 - DNA, 18 bp, Artificial Sequence
  Chemically synthesized
ctgttacagcgtataatg

SEQ ID NO: 116 - DNA, 18 bp, Artificial Sequence
  Chemically synthesized
tgttacagcgtataatgg

SEQ ID NO: 117 - DNA, 18 bp, Artificial Sequence
  Chemically synthesized
gttacagcgtataatggt

SEQ ID NO: 118 - DNA, 18 bp, Artificial Sequence
  Chemically synthesized
ttacagcgtataatggtt

SEQ ID NO: 119 - DNA, 18 bp, Artificial Sequence
  Chemically synthesized
tccacacgcaagttgtgg

SEQ ID NO: 120 - DNA, 18 bp, Artificial Sequence
  Chemically synthesized
agcagacaacccttaagg

SEQ ID NO: 121 - DNA, 18 bp, Artificial Sequence
  Chemically synthesized
gataccttgtacgtgtgg

SEQ ID NO: 122 - DNA, 18 bp, Artificial Sequence
  Chemically synthesized
ataccttgtacgtgtggt

SEQ ID NO: 123 - DNA, 18 bp, Artificial Sequence
  Chemically synthesized
aacgcaagcttcgataat

SEQ ID NO: 124 - DNA, 18 bp, Artificial Sequence
  Chemically synthesized
acgcaagcttcgataatt

SEQ ID NO: 125 - DNA, 18 bp, Artificial Sequence
  Chemically synthesized
gcctgcgaagatctaaaa

SEQ ID NO: 126 - DNA, 18 bp, Artificial Sequence
  Chemically synthesized
ctcaaccgctgctttagg

SEQ ID NO: 127 - DNA, 18 bp, Artificial Sequence
  Chemically synthesized
acaagagtcgaatgtaca

SEQ ID NO: 128 - DNA, 18 bp, Artificial Sequence
  Chemically synthesized
caagagtcgaatgtacaa

SEQ ID NO: 129 - DNA, 18 bp, Artificial Sequence
  Chemically synthesized
cagtcttcttacatcgtt

SEQ ID NO: 130 - DNA, 18 bp, Artificial Sequence
  Chemically synthesized
agtcttcttacatcgttg

SEQ ID NO: 131 - DNA, 18 bp, Artificial Sequence
  Chemically synthesized
gtcttcttacatcgttga

SEQ ID NO: 132 - DNA, 18 bp, Artificial Sequence
  Chemically synthesized
aaatcagcgtctgtttac

SEQ ID NO: 133 - DNA, 18 bp, Artificial Sequence
  Chemically synthesized
tttaacgtaccaatggaa

SEQ ID NO: 134 - DNA, 18 bp, Artificial Sequence
  Chemically synthesized
ttaacgtaccaatggaaa

SEQ ID NO: 135 - DNA, 18 bp, Artificial Sequence
  Chemically synthesized
ttactggcgatagttgta

SEQ ID NO: 136 - DNA, 18 bp, Artificial Sequence
  Chemically synthesized
tactggcgatagttgtaa

SEQ ID NO: 137 - DNA, 18 bp, Artificial Sequence
  Chemically synthesized
actggcgatagttgtaat

SEQ ID NO: 138 - DNA, 18 bp, Artificial Sequence
  Chemically synthesized
tagtgcgcgtcatattaa

SEQ ID NO: 139 - DNA, 18 bp, Artificial Sequence
  Chemically synthesized
catattaatgcgcaggta

SEQ ID NO: 140 - DNA, 18 bp, Artificial Sequence
  Chemically synthesized
atattaatgcgcaggtag

SEQ ID NO: 141 - DNA, 18 bp, Artificial Sequence
  Chemically synthesized
tattaatgcgcaggtagc

SEQ ID NO: 142 - DNA, 18 bp, Artificial Sequence
  Chemically synthesized
attaatgcgcaggtagca

SEQ ID NO: 143 - DNA, 18 bp, Artificial Sequence
  Chemically synthesized
ttaatgcgcaggtagcaa

SEQ ID NO: 144 - DNA, 18 bp, Artificial Sequence
  Chemically synthesized
taatgcgcaggtagcaaa

SEQ ID NO: 145 - DNA, 18 bp, Artificial Sequence
  Chemically synthesized
gtggtgtcactcgtgaca

SEQ ID NO: 146 - DNA, 18 bp, Artificial Sequence
  Chemically synthesized
gtttgcctggcacgatat

SEQ ID NO: 147 - DNA, 18 bp, Artificial Sequence
  Chemically synthesized
cctggcacgatattacgc

SEQ ID NO: 148 - DNA, 18 bp, Artificial Sequence
  Chemically synthesized
ctggcacgatattacgca

SEQ ID NO: 149 - DNA, 18 bp, Artificial Sequence
  Chemically synthesized
tggcacgatattacgcac

SEQ ID NO: 150 - DNA, 18 bp, Artificial Sequence
  Chemically synthesized
ggcacgatattacgcaca

SEQ ID NO: 151 - DNA, 18 bp, Artificial Sequence
  Chemically synthesized
gcacgatattacgcacaa

SEQ ID NO: 152 - DNA, 18 bp, Artificial Sequence
  Chemically synthesized
cacgatattacgcacaac

SEQ ID NO: 153 - DNA, 18 bp, Artificial Sequence
  Chemically synthesized
acgatattacgcacaact

SEQ ID NO: 154 - DNA, 18 bp, Artificial Sequence
  Chemically synthesized
cgatattacgcacaacta

SEQ ID NO: 155 - DNA, 18 bp, Artificial Sequence
  Chemically synthesized
gaaagtttacgccctgac

SEQ ID NO: 156 - DNA, 18 bp, Artificial Sequence
  Chemically synthesized
aaagtttacgccctgaca

SEQ ID NO: 157 - DNA, 18 bp, Artificial Sequence
  Chemically synthesized
aagtttacgccctgacac

SEQ ID NO: 158 - DNA, 18 bp, Artificial Sequence
  Chemically synthesized
agtttacgccctgacaca

SEQ ID NO: 159 - DNA, 18 bp, Artificial Sequence
  Chemically synthesized
gtttacgccctgacacac

SEQ ID NO: 160 - DNA, 18 bp, Artificial Sequence
  Chemically synthesized
tttacgccctgacacacg

SEQ ID NO: 161 - DNA, 18 bp, Artificial Sequence
  Chemically synthesized
ttacgccctgacacacgt

SEQ ID NO: 162 - DNA, 18 bp, Artificial Sequence
  Chemically synthesized
tacgccctgacacacgtt

SEQ ID NO: 163 - DNA, 18 bp, Artificial Sequence
  Chemically synthesized
acgccctgacacacgtta

SEQ ID NO: 164 - DNA, 18 bp, Artificial Sequence
  Chemically synthesized
cgccctgacacacgttat

SEQ ID NO: 165 - DNA, 18 bp, Artificial Sequence
  Chemically synthesized
gccctgacacacgttatg

SEQ ID NO: 166 - DNA, 18 bp, Artificial Sequence
  Chemically synthesized
gagtactgtaggcacggc

SEQ ID NO: 167 - DNA, 18 bp, Artificial Sequence
  Chemically synthesized
gaagctgcgctgtgcacc

SEQ ID NO: 168 - DNA, 18 bp, Artificial Sequence
  Chemically synthesized
atctaaagttgcgtagtg

SEQ ID NO: 169 - DNA, 18 bp, Artificial Sequence
  Chemically synthesized
tctaaagttgcgtagtga

SEQ ID NO: 170 - DNA, 18 bp, Artificial Sequence
  Chemically synthesized
ctattacctcttacgcaa

SEQ ID NO: 171 - DNA, 18 bp, Artificial Sequence
  Chemically synthesized
tattacctcttacgcaat

SEQ ID NO: 172 - DNA, 18 bp, Artificial Sequence
  Chemically synthesized
attacctcttacgcaata

SEQ ID NO: 173 - DNA, 18 bp, Artificial Sequence
  Chemically synthesized
ttacctcttacgcaatat

SEQ ID NO: 174 - DNA, 18 bp, Artificial Sequence
  Chemically synthesized
tacctcttacgcaatata

SEQ ID NO: 175 - DNA, 18 bp, Artificial Sequence
  Chemically synthesized
tcttacgcaatataatag

SEQ ID NO: 176 - DNA, 18 bp, Artificial Sequence
  Chemically synthesized
ttcgtaagtctaatcata

SEQ ID NO: 177 - DNA, 18 bp, Artificial Sequence
  Chemically synthesized
agtttgttcgcattcaac

SEQ ID NO: 178 - DNA, 18 bp, Artificial Sequence
  Chemically synthesized
ggtggtttctcaatcgat

SEQ ID NO: 179 - DNA, 18 bp, Artificial Sequence
  Chemically synthesized
aaactggaattgccgttt

SEQ ID NO: 180 - DNA, 18 bp, Artificial Sequence
  Chemically synthesized
aactggaattgccgtttt

SEQ ID NO: 181 - DNA, 18 bp, Artificial Sequence
  Chemically synthesized
tatgcctgctagttgggt

SEQ ID NO: 182 - DNA, 18 bp, Artificial Sequence
  Chemically synthesized
atgcctgctagttgggtg

SEQ ID NO: 183 - DNA, 18 bp, Artificial Sequence
  Chemically synthesized
gcacatcagtagtcttac

SEQ ID NO: 184 - DNA, 18 bp, Artificial Sequence
  Chemically synthesized
cacatcagtagtcttact

SEQ ID NO: 185 - DNA, 18 bp, Artificial Sequence
  Chemically synthesized
acatcagtagtcttactc

SEQ ID NO: 186 - DNA, 18 bp, Artificial Sequence
  Chemically synthesized
catcagtagtcttactct

SEQ ID NO: 187 - DNA, 18 bp, Artificial Sequence
  Chemically synthesized
atcagtagtcttactctc

SEQ ID NO: 188 - DNA, 18 bp, Artificial Sequence
  Chemically synthesized
atgagcttagtcctgttg

SEQ ID NO: 189 - DNA, 18 bp, Artificial Sequence
  Chemically synthesized
tgagcttagtcctgttgc

SEQ ID NO: 190 - DNA, 18 bp, Artificial Sequence
  Chemically synthesized
ctacgacagatgtcttgt

SEQ ID NO: 191 - DNA, 18 bp, Artificial Sequence
  Chemically synthesized
tgatgacaatgcgttagc

SEQ ID NO: 192 - DNA, 18 bp, Artificial Sequence
  Chemically synthesized
gatgacaatgcgttagct

SEQ ID NO: 193 - DNA, 18 bp, Artificial Sequence
  Chemically synthesized
tgcgttagcttactacaa

SEQ ID NO: 194 - DNA, 18 bp, Artificial Sequence
  Chemically synthesized
ctagctagtgggggacaa

SEQ ID NO: 195 - DNA, 18 bp, Artificial Sequence
  Chemically synthesized
tagctagtgggggacaac

SEQ ID NO: 196 - DNA, 18 bp, Artificial Sequence
  Chemically synthesized
agctagtgggggacaacc

SEQ ID NO: 197 - DNA, 18 bp, Artificial Sequence
  Chemically synthesized
gctagtgggggacaacca

SEQ ID NO: 198 - DNA, 18 bp, Artificial Sequence
  Chemically synthesized
ctagtgggggacaaccaa

SEQ ID NO: 199 - DNA, 18 bp, Artificial Sequence
  Chemically synthesized
tagtgggggacaaccaat

SEQ ID NO: 200 - DNA, 18 bp, Artificial Sequence
  Chemically synthesized
ccgttgccacatagatca

SEQ ID NO: 201 - DNA, 18 bp, Artificial Sequence
  Chemically synthesized
ctgtaccgtctgcggtat

SEQ ID NO: 202 - DNA, 18 bp, Artificial Sequence
  Chemically synthesized
tgtaccgtctgcggtatg

SEQ ID NO: 203 - DNA, 18 bp, Artificial Sequence
  Chemically synthesized
actccgcgaacccatgct

SEQ ID NO: 204 - DNA, 18 bp, Artificial Sequence
  Chemically synthesized
ctccgcgaacccatgctt

SEQ ID NO: 205 - DNA, 18 bp, Artificial Sequence
  Chemically synthesized
cagcccgtcttacaccgt

SEQ ID NO: 206 - DNA, 18 bp, Artificial Sequence
  Chemically synthesized
agcccgtcttacaccgtg

SEQ ID NO: 207 - DNA, 18 bp, Artificial Sequence
  Chemically synthesized
gcccgtcttacaccgtgc

SEQ ID NO: 208 - DNA, 18 bp, Artificial Sequence
  Chemically synthesized
cccgtcttacaccgtgcg

SEQ ID NO: 209 - DNA, 18 bp, Artificial Sequence
  Chemically synthesized
ccgtcttacaccgtgcgg

SEQ ID NO: 210 - DNA, 18 bp, Artificial Sequence
  Chemically synthesized
cgtcttacaccgtgcggc

SEQ ID NO: 211 - DNA, 18 bp, Artificial Sequence
  Chemically synthesized
gtcttacaccgtgcggca

SEQ ID NO: 212 - DNA, 18 bp, Artificial Sequence
  Chemically synthesized
tcttacaccgtgcggcac

SEQ ID NO: 213 - DNA, 18 bp, Artificial Sequence
  Chemically synthesized
cttacaccgtgcggcaca

SEQ ID NO: 214 - DNA, 18 bp, Artificial Sequence
  Chemically synthesized
ttacaccgtgcggcacag

SEQ ID NO: 215 - DNA, 18 bp, Artificial Sequence
  Chemically synthesized
tacaccgtgcggcacagg

SEQ ID NO: 216 - DNA, 18 bp, Artificial Sequence
  Chemically synthesized
acaccgtgcggcacaggc

SEQ ID NO: 217 - DNA, 18 bp, Artificial Sequence
  Chemically synthesized
cggcacaggcactagtac

SEQ ID NO: 218 - DNA, 18 bp, Artificial Sequence
  Chemically synthesized
cgtatacgccaacttagg

SEQ ID NO: 219 - DNA, 18 bp, Artificial Sequence
  Chemically synthesized
gtatacgccaacttaggt

SEQ ID NO: 220 - DNA, 18 bp, Artificial Sequence
  Chemically synthesized
acttaggtgaacgtgtac

SEQ ID NO: 221 - DNA, 18 bp, Artificial Sequence
  Chemically synthesized
cttaggtgaacgtgtacg

SEQ ID NO: 222 - DNA, 18 bp, Artificial Sequence
  Chemically synthesized
gatgccatgcgaaatgct

SEQ ID NO: 223 - DNA, 18 bp, Artificial Sequence
  Chemically synthesized
atgccatgcgaaatgctg

SEQ ID NO: 224 - DNA, 18 bp, Artificial Sequence
  Chemically synthesized
tgccatgcgaaatgctgg

SEQ ID NO: 225 - DNA, 18 bp, Artificial Sequence
  Chemically synthesized
gccatgcgaaatgctggt

SEQ ID NO: 226 - DNA, 18 bp, Artificial Sequence
  Chemically synthesized
ctacagtgttcccaccta

SEQ ID NO: 227 - DNA, 18 bp, Artificial Sequence
  Chemically synthesized
tagctctagacttagttt

SEQ ID NO: 228 - DNA, 18 bp, Artificial Sequence
  Chemically synthesized
atcagcgattatgactac

SEQ ID NO: 229 - DNA, 18 bp, Artificial Sequence
  Chemically synthesized
ttatgactactatcgtta

SEQ ID NO: 230 - DNA, 18 bp, Artificial Sequence
  Chemically synthesized
tatgactactatcgttat

SEQ ID NO: 231 - DNA, 18 bp, Artificial Sequence
  Chemically synthesized
atgactactatcgttata

SEQ ID NO: 232 - DNA, 18 bp, Artificial Sequence
  Chemically synthesized
ttacgatggtggctgtat

SEQ ID NO: 233 - DNA, 18 bp, Artificial Sequence
  Chemically synthesized
ggtaaggctagactttat

SEQ ID NO: 234 - DNA, 18 bp, Artificial Sequence
  Chemically synthesized
aatagccgccactagagg

SEQ ID NO: 235 - DNA, 18 bp, Artificial Sequence
  Chemically synthesized
atagccgccactagagga

SEQ ID NO: 236 - DNA, 18 bp, Artificial Sequence
  Chemically synthesized
aacgtgttgtagcttgtc

SEQ ID NO: 237 - DNA, 18 bp, Artificial Sequence
  Chemically synthesized
acgtgttgtagcttgtca

SEQ ID NO: 238 - DNA, 18 bp, Artificial Sequence
  Chemically synthesized
ttgtagcttgtcacaccg

SEQ ID NO: 239 - DNA, 18 bp, Artificial Sequence
  Chemically synthesized
tgtagcttgtcacaccgt

SEQ ID NO: 240 - DNA, 18 bp, Artificial Sequence
  Chemically synthesized
gtagcttgtcacaccgtt

SEQ ID NO: 241 - DNA, 18 bp, Artificial Sequence
  Chemically synthesized
tagcttgtcacaccgttt

SEQ ID NO: 242 - DNA, 18 bp, Artificial Sequence
  Chemically synthesized
tggcggttcactatatgt

SEQ ID NO: 243 - DNA, 18 bp, Artificial Sequence
  Chemically synthesized
ggcggttcactatatgtt

SEQ ID NO: 244 - DNA, 18 bp, Artificial Sequence
  Chemically synthesized
gcggttcactatatgtta

SEQ ID NO: 245 - DNA, 18 bp, Artificial Sequence
  Chemically synthesized
aaaattgccgataagtat

SEQ ID NO: 246 - DNA, 18 bp, Artificial Sequence
  Chemically synthesized
aaattgccgataagtatg

SEQ ID NO: 247 - DNA, 18 bp, Artificial Sequence
  Chemically synthesized
aattgccgataagtatgt

SEQ ID NO: 248 - DNA, 18 bp, Artificial Sequence
  Chemically synthesized
attgccgataagtatgtc

SEQ ID NO: 249 - DNA, 18 bp, Artificial Sequence
  Chemically synthesized
tacgcatatttgcgtaaa

SEQ ID NO: 250 - DNA, 18 bp, Artificial Sequence
  Chemically synthesized
acgcatatttgcgtaaac

SEQ ID NO: 251 - DNA, 18 bp, Artificial Sequence
  Chemically synthesized
atgatactctctgacgat

SEQ ID NO: 252 - DNA, 18 bp, Artificial Sequence
  Chemically synthesized
tgatactctctgacgatg

SEQ ID NO: 253 - DNA, 18 bp, Artificial Sequence
  Chemically synthesized
gatactctctgacgatgc

SEQ ID NO: 254 - DNA, 18 bp, Artificial Sequence
  Chemically synthesized
atagcacttatgcatctc

SEQ ID NO: 255 - DNA, 18 bp, Artificial Sequence
  Chemically synthesized
tagcacttatgcatctca

SEQ ID NO: 256 - DNA, 18 bp, Artificial Sequence
  Chemically synthesized
atgattgaacggttcgtg

SEQ ID NO: 257 - DNA, 18 bp, Artificial Sequence
  Chemically synthesized
ttgaacggttcgtgtctt

SEQ ID NO: 258 - DNA, 18 bp, Artificial Sequence
  Chemically synthesized
gcatacgtagaccattct

SEQ ID NO: 259 - DNA, 18 bp, Artificial Sequence
  Chemically synthesized
tgttaatccgtatgtttg

SEQ ID NO: 260 - DNA, 18 bp, Artificial Sequence
  Chemically synthesized
gttaatccgtatgtttgc

SEQ ID NO: 261 - DNA, 18 bp, Artificial Sequence
  Chemically synthesized
ttaatccgtatgtttgca

SEQ ID NO: 262 - DNA, 18 bp, Artificial Sequence
  Chemically synthesized
gaggtatgagctattatt

SEQ ID NO: 263 - DNA, 18 bp, Artificial Sequence
  Chemically synthesized
atgtgttggtagcgataa

SEQ ID NO: 264 - DNA, 18 bp, Artificial Sequence
  Chemically synthesized
tgtgttggtagcgataat

SEQ ID NO: 265 - DNA, 18 bp, Artificial Sequence
  Chemically synthesized
gtgttggtagcgataatg

SEQ ID NO: 266 - DNA, 18 bp, Artificial Sequence
  Chemically synthesized
gcagaaacgctcaaagct

SEQ ID NO: 267 - DNA, 18 bp, Artificial Sequence
  Chemically synthesized
ggtattgctactgtacgt

SEQ ID NO: 268 - DNA, 18 bp, Artificial Sequence
  Chemically synthesized
ctactgtacgtgaagtgc

SEQ ID NO: 269 - DNA, 18 bp, Artificial Sequence
  Chemically synthesized
gtacgtgaagtgctgtct

SEQ ID NO: 270 - DNA, 18 bp, Artificial Sequence
  Chemically synthesized
ccaccacttaaccgaaat

SEQ ID NO: 271 - DNA, 18 bp, Artificial Sequence
  Chemically synthesized
caccacttaaccgaaatt

SEQ ID NO: 272 - DNA, 18 bp, Artificial Sequence
  Chemically synthesized
accacttaaccgaaatta

SEQ ID NO: 273 - DNA, 18 bp, Artificial Sequence
  Chemically synthesized
ccacttaaccgaaattat

SEQ ID NO: 274 - DNA, 18 bp, Artificial Sequence
  Chemically synthesized
cacttaaccgaaattatg

SEQ ID NO: 275 - DNA, 18 bp, Artificial Sequence
  Chemically synthesized
acttaaccgaaattatgt

SEQ ID NO: 276 - DNA, 18 bp, Artificial Sequence
  Chemically synthesized
cttaaccgaaattatgtc

SEQ ID NO: 277 - DNA, 18 bp, Artificial Sequence
  Chemically synthesized
ttaaccgaaattatgtct

SEQ ID NO: 278 - DNA, 18 bp, Artificial Sequence
  Chemically synthesized
ggttatcgtgtaactaaa

SEQ ID NO: 279 - DNA, 18 bp, Artificial Sequence
  Chemically synthesized
gttgtttaccgaggtaca

SEQ ID NO: 280 - DNA, 18 bp, Artificial Sequence
  Chemically synthesized
ttgtttaccgaggtacaa

SEQ ID NO: 281 - DNA, 18 bp, Artificial Sequence
  Chemically synthesized
gtgccacaagagcactat

SEQ ID NO: 282 - DNA, 18 bp, Artificial Sequence
  Chemically synthesized
gctctcatgccgctgttg

SEQ ID NO: 283 - DNA, 18 bp, Artificial Sequence
  Chemically synthesized
ctctcatgccgctgttga

SEQ ID NO: 284 - DNA, 18 bp, Artificial Sequence
  Chemically synthesized
tctcatgccgctgttgat

SEQ ID NO: 285 - DNA, 18 bp, Artificial Sequence
  Chemically synthesized
ctcatgccgctgttgatg

SEQ ID NO: 286 - DNA, 18 bp, Artificial Sequence
  Chemically synthesized
gcattgcctgagacgaca

SEQ ID NO: 287 - DNA, 18 bp, Artificial Sequence
  Chemically synthesized
cattgcctgagacgacag

SEQ ID NO: 288 - DNA, 18 bp, Artificial Sequence
  Chemically synthesized
attgcctgagacgacagc

SEQ ID NO: 289 - DNA, 18 bp, Artificial Sequence
  Chemically synthesized
atgccagattacgtgcta

SEQ ID NO: 290 - DNA, 18 bp, Artificial Sequence
  Chemically synthesized
cagattacgtgctaagca

SEQ ID NO: 291 - DNA, 18 bp, Artificial Sequence
  Chemically synthesized
tgtacattggcgaccctg

SEQ ID NO: 292 - DNA, 18 bp, Artificial Sequence
  Chemically synthesized
taagggtgttatcacgca

SEQ ID NO: 293 - DNA, 18 bp, Artificial Sequence
  Chemically synthesized
aagggtgttatcacgcat

SEQ ID NO: 294 - DNA, 18 bp, Artificial Sequence
  Chemically synthesized
ggccacaaataggcgtgg

SEQ ID NO: 295 - DNA, 18 bp, Artificial Sequence
  Chemically synthesized
attccttacacgtaaccc

SEQ ID NO: 296 - DNA, 18 bp, Artificial Sequence
  Chemically synthesized
ttccttacacgtaaccct

SEQ ID NO: 297 - DNA, 18 bp, Artificial Sequence
  Chemically synthesized
cgtaaccctgcttggaga

SEQ ID NO: 298 - DNA, 18 bp, Artificial Sequence
  Chemically synthesized
attccacgtaggaatgtg

SEQ ID NO: 299 - DNA, 18 bp, Artificial Sequence
  Chemically synthesized
ttccacgtaggaatgtgg

SEQ ID NO: 300 - DNA, 18 bp, Artificial Sequence
  Chemically synthesized
tccacgtaggaatgtggc

SEQ ID NO: 301 - DNA, 18 bp, Artificial Sequence
  Chemically synthesized
ccacgtaggaatgtggca

SEQ ID NO: 302 - DNA, 18 bp, Artificial Sequence
  Chemically synthesized
cacgtaggaatgtggcaa

SEQ ID NO: 303 - DNA, 18 bp, Artificial Sequence
  Chemically synthesized
acgtaggaatgtggcaac

SEQ ID NO: 304 - DNA, 18 bp, Artificial Sequence
  Chemically synthesized
ctctatgatgggttttaa

SEQ ID NO: 305 - DNA, 18 bp, Artificial Sequence
  Chemically synthesized
acatgtacgtgcatggat

SEQ ID NO: 306 - DNA, 18 bp, Artificial Sequence
  Chemically synthesized
catgtacgtgcatggatt

SEQ ID NO: 307 - DNA, 18 bp, Artificial Sequence
  Chemically synthesized
caggtgttaacctagttg

SEQ ID NO: 308 - DNA, 18 bp, Artificial Sequence
  Chemically synthesized
aggtgttaacctagttgc

SEQ ID NO: 309 - DNA, 18 bp, Artificial Sequence
  Chemically synthesized
ggaatgtagtgcgtataa

SEQ ID NO: 310 - DNA, 18 bp, Artificial Sequence
  Chemically synthesized
gaatgtagtgcgtataaa

SEQ ID NO: 311 - DNA, 18 bp, Artificial Sequence
  Chemically synthesized
aatgtagtgcgtataaag

SEQ ID NO: 312 - DNA, 18 bp, Artificial Sequence
  Chemically synthesized
atgtagtgcgtataaaga

SEQ ID NO: 313 - DNA, 18 bp, Artificial Sequence
  Chemically synthesized
tgtagtgcgtataaagat

SEQ ID NO: 314 - DNA, 18 bp, Artificial Sequence
  Chemically synthesized
ctctgacagagtcgtatt

SEQ ID NO: 315 - DNA, 18 bp, Artificial Sequence
  Chemically synthesized
tctgacagagtcgtattt

SEQ ID NO: 316 - DNA, 18 bp, Artificial Sequence
  Chemically synthesized
ctgacagagtcgtatttg

SEQ ID NO: 317 - DNA, 18 bp, Artificial Sequence
  Chemically synthesized
ggacctgagcgcacctgt

SEQ ID NO: 318 - DNA, 18 bp, Artificial Sequence
  Chemically synthesized
gacctgagcgcacctgtt

SEQ ID NO: 319 - DNA, 18 bp, Artificial Sequence
  Chemically synthesized
acctgagcgcacctgttg

SEQ ID NO: 320 - DNA, 18 bp, Artificial Sequence
  Chemically synthesized
ttacgtctataatccgtt

SEQ ID NO: 321 - DNA, 18 bp, Artificial Sequence
  Chemically synthesized
tacgtctataatccgttt

SEQ ID NO: 322 - DNA, 18 bp, Artificial Sequence
  Chemically synthesized
acgtctataatccgttta

SEQ ID NO: 323 - DNA, 18 bp, Artificial Sequence
  Chemically synthesized
cgtctataatccgtttat

SEQ ID NO: 324 - DNA, 18 bp, Artificial Sequence
  Chemically synthesized
gtctataatccgtttatg

SEQ ID NO: 325 - DNA, 18 bp, Artificial Sequence
  Chemically synthesized
tctataatccgtttatga

SEQ ID NO: 326 - DNA, 18 bp, Artificial Sequence
  Chemically synthesized
taatgcggcttgtagaaa

SEQ ID NO: 327 - DNA, 18 bp, Artificial Sequence
  Chemically synthesized
aatgcggcttgtagaaag

SEQ ID NO: 328 - DNA, 18 bp, Artificial Sequence
  Chemically synthesized
atgcggcttgtagaaagg

SEQ ID NO: 329 - DNA, 18 bp, Artificial Sequence
  Chemically synthesized
tgcggcttgtagaaaggt

SEQ ID NO: 330 - DNA, 18 bp, Artificial Sequence
  Chemically synthesized
attgcaatgtcgatagat

SEQ ID NO: 331 - DNA, 18 bp, Artificial Sequence
  Chemically synthesized
ttgcaatgtcgatagata

SEQ ID NO: 332 - DNA, 18 bp, Artificial Sequence
  Chemically synthesized
acactagagtgctatcta

SEQ ID NO: 333 - DNA, 18 bp, Artificial Sequence
  Chemically synthesized
ctgctacgtgtataacac

SEQ ID NO: 334 - DNA, 18 bp, Artificial Sequence
  Chemically synthesized
gagctttgggctaagcgc

SEQ ID NO: 335 - DNA, 18 bp, Artificial Sequence
  Chemically synthesized
agctttgggctaagcgca

SEQ ID NO: 336 - DNA, 18 bp, Artificial Sequence
  Chemically synthesized
gctttgggctaagcgcaa

SEQ ID NO: 337 - DNA, 18 bp, Artificial Sequence
  Chemically synthesized
ctttgggctaagcgcaac

SEQ ID NO: 338 - DNA, 18 bp, Artificial Sequence
  Chemically synthesized
tttgggctaagcgcaaca

SEQ ID NO: 339 - DNA, 18 bp, Artificial Sequence
  Chemically synthesized
ttgggctaagcgcaacat

SEQ ID NO: 340 - DNA, 18 bp, Artificial Sequence
  Chemically synthesized
tgggctaagcgcaacatt

SEQ ID NO: 341 - DNA, 18 bp, Artificial Sequence
  Chemically synthesized
gggctaagcgcaacatta

SEQ ID NO: 342 - DNA, 18 bp, Artificial Sequence
  Chemically synthesized
ggctaagcgcaacattaa

SEQ ID NO: 343 - DNA, 18 bp, Artificial Sequence
  Chemically synthesized
atggtcaagtagacttat

SEQ ID NO: 344 - DNA, 18 bp, Artificial Sequence
  Chemically synthesized
gaaatgcccgtaatggtg

SEQ ID NO: 345 - DNA, 18 bp, Artificial Sequence
  Chemically synthesized
ccttcgaacatatcgttt

SEQ ID NO: 346 - DNA, 18 bp, Artificial Sequence
  Chemically synthesized
cttcgaacatatcgttta

SEQ ID NO: 347 - DNA, 18 bp, Artificial Sequence
  Chemically synthesized
ttcgaacatatcgtttat

SEQ ID NO: 348 - DNA, 18 bp, Artificial Sequence
  Chemically synthesized
tcgaacatatcgtttatg

SEQ ID NO: 349 - DNA, 18 bp, Artificial Sequence
  Chemically synthesized
gtcatagtcagttaggtg

SEQ ID NO: 350 - DNA, 18 bp, Artificial Sequence
  Chemically synthesized
tcatagtcagttaggtgg

SEQ ID NO: 351 - DNA, 18 bp, Artificial Sequence
  Chemically synthesized
catagtcagttaggtggt

SEQ ID NO: 352 - DNA, 18 bp, Artificial Sequence
  Chemically synthesized
attggactagctaaacgt

SEQ ID NO: 353 - DNA, 18 bp, Artificial Sequence
  Chemically synthesized
ttggactagctaaacgtt

SEQ ID NO: 354 - DNA, 18 bp, Artificial Sequence
  Chemically synthesized
tacaatctagtcaagcgt

SEQ ID NO: 355 - DNA, 18 bp, Artificial Sequence
  Chemically synthesized
acaatctagtcaagcgtg

SEQ ID NO: 356 - DNA, 18 bp, Artificial Sequence
  Chemically synthesized
caatctagtcaagcgtgg

SEQ ID NO: 357 - DNA, 18 bp, Artificial Sequence
  Chemically synthesized
agcgtggcaaccgggtgt

SEQ ID NO: 358 - DNA, 18 bp, Artificial Sequence
  Chemically synthesized
gcgtggcaaccgggtgtt

SEQ ID NO: 359 - DNA, 18 bp, Artificial Sequence
  Chemically synthesized
ggagttgcaccaggtaca

SEQ ID NO: 360 - DNA, 18 bp, Artificial Sequence
  Chemically synthesized
tacgaccctaagactaaa

SEQ ID NO: 361 - DNA, 18 bp, Artificial Sequence
  Chemically synthesized
tgggacacttcgcatggt

SEQ ID NO: 362 - DNA, 18 bp, Artificial Sequence
  Chemically synthesized
gggacacttcgcatggtg

SEQ ID NO: 363 - DNA, 18 bp, Artificial Sequence
  Chemically synthesized
ggacacttcgcatggtgg

SEQ ID NO: 364 - DNA, 18 bp, Artificial Sequence
  Chemically synthesized
gacacttcgcatggtgga

SEQ ID NO: 365 - DNA, 18 bp, Artificial Sequence
  Chemically synthesized
acacttcgcatggtggac

SEQ ID NO: 366 - DNA, 18 bp, Artificial Sequence
  Chemically synthesized
actaatgtgaatgcgtca

SEQ ID NO: 367 - DNA, 18 bp, Artificial Sequence
  Chemically synthesized
ctaatgtgaatgcgtcat

SEQ ID NO: 368 - DNA, 18 bp, Artificial Sequence
  Chemically synthesized
taatgtgaatgcgtcatc

SEQ ID NO: 369 - DNA, 18 bp, Artificial Sequence
  Chemically synthesized
aatgtgaatgcgtcatca

SEQ ID NO: 370 - DNA, 18 bp, Artificial Sequence
  Chemically synthesized
atcttggcaaaccacgcg

SEQ ID NO: 371 - DNA, 18 bp, Artificial Sequence
  Chemically synthesized
tcttggcaaaccacgcga

SEQ ID NO: 372 - DNA, 18 bp, Artificial Sequence
  Chemically synthesized
cttggcaaaccacgcgaa

SEQ ID NO: 373 - DNA, 18 bp, Artificial Sequence
  Chemically synthesized
caaaccacgcgaacaaat

SEQ ID NO: 374 - DNA, 18 bp, Artificial Sequence
  Chemically synthesized
aaaccacgcgaacaaata

SEQ ID NO: 375 - DNA, 18 bp, Artificial Sequence
  Chemically synthesized
aaccacgcgaacaaatag

SEQ ID NO: 376 - DNA, 18 bp, Artificial Sequence
  Chemically synthesized
accacgcgaacaaataga

SEQ ID NO: 377 - DNA, 18 bp, Artificial Sequence
  Chemically synthesized
ttgttaataacgctacta

SEQ ID NO: 378 - DNA, 18 bp, Artificial Sequence
  Chemically synthesized
tattctaagcacacgcct

SEQ ID NO: 379 - DNA, 18 bp, Artificial Sequence
  Chemically synthesized
attctaagcacacgccta

SEQ ID NO: 380 - DNA, 18 bp, Artificial Sequence
  Chemically synthesized
ttaatttagtgcgtgatc

SEQ ID NO: 381 - DNA, 18 bp, Artificial Sequence
  Chemically synthesized
taatttagtgcgtgatct

SEQ ID NO: 382 - DNA, 18 bp, Artificial Sequence
  Chemically synthesized
aatttagtgcgtgatctc

SEQ ID NO: 383 - DNA, 18 bp, Artificial Sequence
  Chemically synthesized
atttagtgcgtgatctcc

SEQ ID NO: 384 - DNA, 18 bp, Artificial Sequence
  Chemically synthesized
tttagtgcgtgatctccc

SEQ ID NO: 385 - DNA, 18 bp, Artificial Sequence
  Chemically synthesized
aaagtgtacgttgaaatc

SEQ ID NO: 386 - DNA, 18 bp, Artificial Sequence
  Chemically synthesized
atcaggccggtagcacac

SEQ ID NO: 387 - DNA, 18 bp, Artificial Sequence
  Chemically synthesized
tcaggccggtagcacacc

SEQ ID NO: 388 - DNA, 18 bp, Artificial Sequence
  Chemically synthesized
caggccggtagcacacct

SEQ ID NO: 389 - DNA, 18 bp, Artificial Sequence
  Chemically synthesized
aggccggtagcacacctt

SEQ ID NO: 390 - DNA, 18 bp, Artificial Sequence
  Chemically synthesized
ggccggtagcacaccttg

SEQ ID NO: 391 - DNA, 18 bp, Artificial Sequence
  Chemically synthesized
gccggtagcacaccttgt

SEQ ID NO: 392 - DNA, 18 bp, Artificial Sequence
  Chemically synthesized
gttcttactgagtctaac

SEQ ID NO: 393 - DNA, 18 bp, Artificial Sequence
  Chemically synthesized
ttcttactgagtctaaca

SEQ ID NO: 394 - DNA, 18 bp, Artificial Sequence
  Chemically synthesized
ggtgcaggtatatgcgct

SEQ ID NO: 395 - DNA, 18 bp, Artificial Sequence
  Chemically synthesized
gtgcaggtatatgcgcta

SEQ ID NO: 396 - DNA, 18 bp, Artificial Sequence
  Chemically synthesized
tgcaggtatatgcgctag

SEQ ID NO: 397 - DNA, 18 bp, Artificial Sequence
  Chemically synthesized
gcgctagttatcagactc

SEQ ID NO: 398 - DNA, 18 bp, Artificial Sequence
  Chemically synthesized
ttaaaccgtgctttaact

SEQ ID NO: 399 - DNA, 18 bp, Artificial Sequence
  Chemically synthesized
cacaaaagtttaacggcc

SEQ ID NO: 400 - DNA, 18 bp, Artificial Sequence
  Chemically synthesized
acaaaagtttaacggcct

SEQ ID NO: 401 - DNA, 18 bp, Artificial Sequence
  Chemically synthesized
caaaagtttaacggcctt

SEQ ID NO: 402 - DNA, 18 bp, Artificial Sequence
  Chemically synthesized
aaaagtttaacggcctta

SEQ ID NO: 403 - DNA, 18 bp, Artificial Sequence
  Chemically synthesized
aaagtttaacggccttac

SEQ ID NO: 404 - DNA, 18 bp, Artificial Sequence
  Chemically synthesized
aagtttaacggccttact

SEQ ID NO: 405 - DNA, 18 bp, Artificial Sequence
  Chemically synthesized
agtttaacggccttactg

SEQ ID NO: 406 - DNA, 18 bp, Artificial Sequence
  Chemically synthesized
gtttaacggccttactgt

SEQ ID NO: 407 - DNA, 18 bp, Artificial Sequence
  Chemically synthesized
tttaacggccttactgtt

SEQ ID NO: 408 - DNA, 18 bp, Artificial Sequence
  Chemically synthesized
tggttggacctttggtgc

SEQ ID NO: 409 - DNA, 18 bp, Artificial Sequence
  Chemically synthesized
actttcctcgtgaaggtg

SEQ ID NO: 410 - DNA, 18 bp, Artificial Sequence
  Chemically synthesized
aaattgaccgcctcaatg

SEQ ID NO: 411 - DNA, 18 bp, Artificial Sequence
  Chemically synthesized
aattgaccgcctcaatga

SEQ ID NO: 412 - DNA, 18 bp, Artificial Sequence
  Chemically synthesized
attgaccgcctcaatgag

SEQ ID NO: 413 - DNA, 18 bp, Artificial Sequence
  Chemically synthesized
ttgaccgcctcaatgagg

SEQ ID NO: 414 - DNA, 18 bp, Artificial Sequence
  Chemically synthesized
tgaccgcctcaatgaggt

SEQ ID NO: 415 - DNA, 18 bp, Artificial Sequence
  Chemically synthesized
gaccgcctcaatgaggtt

SEQ ID NO: 416 - DNA, 18 bp, Artificial Sequence
  Chemically synthesized
accgcctcaatgaggttg

SEQ ID NO: 417 - DNA, 18 bp, Artificial Sequence
  Chemically synthesized
agacgactctgagccagt

SEQ ID NO: 418 - DNA, 18 bp, Artificial Sequence
  Chemically synthesized
attttgttcgcgctactg

SEQ ID NO: 419 - DNA, 18 bp, Artificial Sequence
  Chemically synthesized
ttttgttcgcgctactgc

SEQ ID NO: 420 - DNA, 18 bp, Artificial Sequence
  Chemically synthesized
gttcgcgctactgcaacg

SEQ ID NO: 421 - DNA, 18 bp, Artificial Sequence
  Chemically synthesized
ttcgcgctactgcaacga

SEQ ID NO: 422 - DNA, 18 bp, Artificial Sequence
  Chemically synthesized
tcgcgctactgcaacgat

SEQ ID NO: 423 - DNA, 18 bp, Artificial Sequence
  Chemically synthesized
tttcggatggcttattgt

SEQ ID NO: 424 - DNA, 18 bp, Artificial Sequence
  Chemically synthesized
ttcggatggcttattgtt

SEQ ID NO: 425 - DNA, 18 bp, Artificial Sequence
  Chemically synthesized
tcggatggcttattgttg

SEQ ID NO: 426 - DNA, 18 bp, Artificial Sequence
  Chemically synthesized
atggcttattgttggcgt

SEQ ID NO: 427 - DNA, 18 bp, Artificial Sequence
  Chemically synthesized
tggcttattgttggcgtt

SEQ ID NO: 428 - DNA, 18 bp, Artificial Sequence
  Chemically synthesized
tcacacaatcgacggttc

SEQ ID NO: 429 - DNA, 18 bp, Artificial Sequence
  Chemically synthesized
cacacaatcgacggttca

SEQ ID NO: 430 - DNA, 18 bp, Artificial Sequence
  Chemically synthesized
acacaatcgacggttcat

SEQ ID NO: 431 - DNA, 18 bp, Artificial Sequence
  Chemically synthesized
cacaatcgacggttcatc

SEQ ID NO: 432 - DNA, 18 bp, Artificial Sequence
  Chemically synthesized
tatgatgaaccgacgacg

SEQ ID NO: 433 - DNA, 18 bp, Artificial Sequence
  Chemically synthesized
atgatgaaccgacgacga

SEQ ID NO: 434 - DNA, 18 bp, Artificial Sequence
  Chemically synthesized
acaagctgatgagtacga

SEQ ID NO: 435 - DNA, 18 bp, Artificial Sequence
  Chemically synthesized
caagctgatgagtacgaa

SEQ ID NO: 436 - DNA, 18 bp, Artificial Sequence
  Chemically synthesized
aagctgatgagtacgaac

SEQ ID NO: 437 - DNA, 18 bp, Artificial Sequence
  Chemically synthesized
agctgatgagtacgaact

SEQ ID NO: 438 - DNA, 18 bp, Artificial Sequence
  Chemically synthesized
gctgatgagtacgaactt

SEQ ID NO: 439 - DNA, 18 bp, Artificial Sequence
  Chemically synthesized
ctgatgagtacgaactta

SEQ ID NO: 440 - DNA, 18 bp, Artificial Sequence
  Chemically synthesized
tgatgagtacgaacttat

SEQ ID NO: 441 - DNA, 18 bp, Artificial Sequence
  Chemically synthesized
gatgagtacgaacttatg

SEQ ID NO: 442 - DNA, 18 bp, Artificial Sequence
  Chemically synthesized
gagacaggtacgttaata

SEQ ID NO: 443 - DNA, 18 bp, Artificial Sequence
  Chemically synthesized
agacaggtacgttaatag

SEQ ID NO: 444 - DNA, 18 bp, Artificial Sequence
  Chemically synthesized
gacaggtacgttaatagt

SEQ ID NO: 445 - DNA, 18 bp, Artificial Sequence
  Chemically synthesized
aatagcgtacttcttttt

SEQ ID NO: 446 - DNA, 18 bp, Artificial Sequence
  Chemically synthesized
gcgcttcgattgtgtgcg

SEQ ID NO: 447 - DNA, 18 bp, Artificial Sequence
  Chemically synthesized
cgcttcgattgtgtgcgt

SEQ ID NO: 448 - DNA, 18 bp, Artificial Sequence
  Chemically synthesized
gcttcgattgtgtgcgta

SEQ ID NO: 449 - DNA, 18 bp, Artificial Sequence
  Chemically synthesized
cttcgattgtgtgcgtac

SEQ ID NO: 450 - DNA, 18 bp, Artificial Sequence
  Chemically synthesized
ttcgattgtgtgcgtact

SEQ ID NO: 451 - DNA, 18 bp, Artificial Sequence
  Chemically synthesized
tttacgtttactctcgtg

SEQ ID NO: 452 - DNA, 18 bp, Artificial Sequence
  Chemically synthesized
ttacgtttactctcgtgt

SEQ ID NO: 453 - DNA, 18 bp, Artificial Sequence
  Chemically synthesized
ggcagattccaacggtac

SEQ ID NO: 454 - DNA, 18 bp, Artificial Sequence
  Chemically synthesized
gcagattccaacggtact

SEQ ID NO: 455 - DNA, 18 bp, Artificial Sequence
  Chemically synthesized
cagattccaacggtacta

SEQ ID NO: 456 - DNA, 18 bp, Artificial Sequence
  Chemically synthesized
agattccaacggtactat

SEQ ID NO: 457 - DNA, 18 bp, Artificial Sequence
  Chemically synthesized
gattccaacggtactatt

SEQ ID NO: 458 - DNA, 18 bp, Artificial Sequence
  Chemically synthesized
ctattaccgttgaagagc

SEQ ID NO: 459 - DNA, 18 bp, Artificial Sequence
  Chemically synthesized
tattaccgttgaagagct

SEQ ID NO: 460 - DNA, 18 bp, Artificial Sequence
  Chemically synthesized
gcgcgtacgcgttccatg

SEQ ID NO: 461 - DNA, 18 bp, Artificial Sequence
  Chemically synthesized
cgcgtacgcgttccatgt

SEQ ID NO: 462 - DNA, 18 bp, Artificial Sequence
  Chemically synthesized
tattctgaccagaccgct

SEQ ID NO: 463 - DNA, 18 bp, Artificial Sequence
  Chemically synthesized
attctgaccagaccgctt

SEQ ID NO: 464 - DNA, 18 bp, Artificial Sequence
  Chemically synthesized
aagtgaactcgtaatcgg

SEQ ID NO: 465 - DNA, 18 bp, Artificial Sequence
  Chemically synthesized
agtgaactcgtaatcgga

SEQ ID NO: 466 - DNA, 18 bp, Artificial Sequence
  Chemically synthesized
gtgaactcgtaatcggag

SEQ ID NO: 467 - DNA, 18 bp, Artificial Sequence
  Chemically synthesized
tgaactcgtaatcggagc

SEQ ID NO: 468 - DNA, 18 bp, Artificial Sequence
  Chemically synthesized
gaactcgtaatcggagct

SEQ ID NO: 469 - DNA, 18 bp, Artificial Sequence
  Chemically synthesized
aactcgtaatcggagctg

SEQ ID NO: 470 - DNA, 18 bp, Artificial Sequence
  Chemically synthesized
actcgtaatcggagctgt

SEQ ID NO: 471 - DNA, 18 bp, Artificial Sequence
  Chemically synthesized
ctcgtaatcggagctgtg

SEQ ID NO: 472 - DNA, 18 bp, Artificial Sequence
  Chemically synthesized
tcgtaatcggagctgtga

SEQ ID NO: 473 - DNA, 18 bp, Artificial Sequence
  Chemically synthesized
cgtaatcggagctgtgat

SEQ ID NO: 474 - DNA, 18 bp, Artificial Sequence
  Chemically synthesized
gtaatcggagctgtgatc

SEQ ID NO: 475 - DNA, 18 bp, Artificial Sequence
  Chemically synthesized
taatcggagctgtgatcc

SEQ ID NO: 476 - DNA, 18 bp, Artificial Sequence
  Chemically synthesized
ctgttgctacatcacgaa

SEQ ID NO: 477 - DNA, 18 bp, Artificial Sequence
  Chemically synthesized
tgttgctacatcacgaac

SEQ ID NO: 478 - DNA, 18 bp, Artificial Sequence
  Chemically synthesized
gttgctacatcacgaacg

SEQ ID NO: 479 - DNA, 18 bp, Artificial Sequence
  Chemically synthesized
ttgctacatcacgaacgc

SEQ ID NO: 480 - DNA, 18 bp, Artificial Sequence
  Chemically synthesized
tgctacatcacgaacgct

SEQ ID NO: 481 - DNA, 18 bp, Artificial Sequence
  Chemically synthesized
gctacatcacgaacgctt

SEQ ID NO: 482 - DNA, 18 bp, Artificial Sequence
  Chemically synthesized
ctacatcacgaacgcttt

SEQ ID NO: 483 - DNA, 18 bp, Artificial Sequence
  Chemically synthesized
caaattgggagcttcgca

SEQ ID NO: 484 - DNA, 18 bp, Artificial Sequence
  Chemically synthesized
ttgctgcatacagtcgct

SEQ ID NO: 485 - DNA, 18 bp, Artificial Sequence
  Chemically synthesized
tgctgcatacagtcgcta

SEQ ID NO: 486 - DNA, 18 bp, Artificial Sequence
  Chemically synthesized
gctgcatacagtcgctac

SEQ ID NO: 487 - DNA, 18 bp, Artificial Sequence
  Chemically synthesized
ctgcatacagtcgctaca

SEQ ID NO: 488 - DNA, 18 bp, Artificial Sequence
  Chemically synthesized
tgcatacagtcgctacag

SEQ ID NO: 489 - DNA, 18 bp, Artificial Sequence
  Chemically synthesized
ggagattgattaaacgaa

SEQ ID NO: 490 - DNA, 18 bp, Artificial Sequence
  Chemically synthesized
gataacactcgctacttg

SEQ ID NO: 491 - DNA, 18 bp, Artificial Sequence
  Chemically synthesized
ataacactcgctacttgt

SEQ ID NO: 492 - DNA, 18 bp, Artificial Sequence
  Chemically synthesized
taacactcgctacttgtg

SEQ ID NO: 493 - DNA, 18 bp, Artificial Sequence
  Chemically synthesized
aacactcgctacttgtga

SEQ ID NO: 494 - DNA, 18 bp, Artificial Sequence
  Chemically synthesized
acactcgctacttgtgag

SEQ ID NO: 495 - DNA, 18 bp, Artificial Sequence
  Chemically synthesized
cactcgctacttgtgagc

SEQ ID NO: 496 - DNA, 18 bp, Artificial Sequence
  Chemically synthesized
actcgctacttgtgagct

SEQ ID NO: 497 - DNA, 18 bp, Artificial Sequence
  Chemically synthesized
ctcgctacttgtgagctt

SEQ ID NO: 498 - DNA, 18 bp, Artificial Sequence
  Chemically synthesized
tacgagggcaattcacca

SEQ ID NO: 499 - DNA, 18 bp, Artificial Sequence
  Chemically synthesized
tcctgacggcgtaaaaca

SEQ ID NO: 500 - DNA, 18 bp, Artificial Sequence
  Chemically synthesized
cctgacggcgtaaaacac

SEQ ID NO: 501 - DNA, 18 bp, Artificial Sequence
  Chemically synthesized
ctgacggcgtaaaacacg

SEQ ID NO: 502 - DNA, 18 bp, Artificial Sequence
  Chemically synthesized
tgacggcgtaaaacacgt

SEQ ID NO: 503 - DNA, 18 bp, Artificial Sequence
  Chemically synthesized
gtctatcagttacgtgcc

SEQ ID NO: 504 - DNA, 18 bp, Artificial Sequence
  Chemically synthesized
tctatcagttacgtgcca

SEQ ID NO: 505 - DNA, 18 bp, Artificial Sequence
  Chemically synthesized
ctatcagttacgtgccag

SEQ ID NO: 506 - DNA, 18 bp, Artificial Sequence
  Chemically synthesized
atatcggtaattatacag

SEQ ID NO: 507 - DNA, 18 bp, Artificial Sequence
  Chemically synthesized
tatcggtaattatacagt

SEQ ID NO: 508 - DNA, 18 bp, Artificial Sequence
  Chemically synthesized
atcggtaattatacagtt

SEQ ID NO: 509 - DNA, 18 bp, Artificial Sequence
  Chemically synthesized
ttagagtatcatgacgtt

SEQ ID NO: 510 - DNA, 18 bp, Artificial Sequence
  Chemically synthesized
tagagtatcatgacgttc

SEQ ID NO: 511 - DNA, 18 bp, Artificial Sequence
  Chemically synthesized
agagtatcatgacgttcg

SEQ ID NO: 512 - DNA, 18 bp, Artificial Sequence
  Chemically synthesized
gagtatcatgacgttcgt

SEQ ID NO: 513 - DNA, 18 bp, Artificial Sequence
  Chemically synthesized
cattacgtttggtggacc

SEQ ID NO: 514 - DNA, 18 bp, Artificial Sequence
  Chemically synthesized
gtcttggttcaccgctct

SEQ ID NO: 515 - DNA, 18 bp, Artificial Sequence
  Chemically synthesized
tcttggttcaccgctctc

SEQ ID NO: 516 - DNA, 18 bp, Artificial Sequence
  Chemically synthesized
caccgctctcactcaaca

SEQ ID NO: 517 - DNA, 18 bp, Artificial Sequence
  Chemically synthesized
accgctctcactcaacat

SEQ ID NO: 518 - DNA, 18 bp, Artificial Sequence
  Chemically synthesized
caaattggctactaccga

SEQ ID NO: 519 - DNA, 18 bp, Artificial Sequence
  Chemically synthesized
aaattggctactaccgaa

SEQ ID NO: 520 - DNA, 18 bp, Artificial Sequence
  Chemically synthesized
aattggctactaccgaag

SEQ ID NO: 521 - DNA, 18 bp, Artificial Sequence
  Chemically synthesized
attggctactaccgaaga

SEQ ID NO: 522 - DNA, 18 bp, Artificial Sequence
  Chemically synthesized
ttggctactaccgaagag

SEQ ID NO: 523 - DNA, 18 bp, Artificial Sequence
  Chemically synthesized
tggctactaccgaagagc

SEQ ID NO: 524 - DNA, 18 bp, Artificial Sequence
  Chemically synthesized
tactaccgaagagctacc

SEQ ID NO: 525 - DNA, 18 bp, Artificial Sequence
  Chemically synthesized
tcatatgggttgcaactg

SEQ ID NO: 526 - DNA, 18 bp, Artificial Sequence
  Chemically synthesized
catatgggttgcaactga

SEQ ID NO: 527 - DNA, 18 bp, Artificial Sequence
  Chemically synthesized
tcacattggcacccgcaa

SEQ ID NO: 528 - DNA, 18 bp, Artificial Sequence
  Chemically synthesized
cacattggcacccgcaat

SEQ ID NO: 529 - DNA, 18 bp, Artificial Sequence
  Chemically synthesized
ggcggcagtcaagcctct

SEQ ID NO: 530 - DNA, 18 bp, Artificial Sequence
  Chemically synthesized
gcggcagtcaagcctctt

SEQ ID NO: 531 - DNA, 18 bp, Artificial Sequence
  Chemically synthesized
cacgtagtcgcaacagtt

SEQ ID NO: 532 - DNA, 18 bp, Artificial Sequence
  Chemically synthesized
acgtagtcgcaacagttc

SEQ ID NO: 533 - DNA, 18 bp, Artificial Sequence
  Chemically synthesized
gcttcagcgttcttcgga

SEQ ID NO: 534 - DNA, 18 bp, Artificial Sequence
  Chemically synthesized
cttcagcgttcttcggaa

SEQ ID NO: 535 - DNA, 18 bp, Artificial Sequence
  Chemically synthesized
ttcagcgttcttcggaat

SEQ ID NO: 536 - DNA, 18 bp, Artificial Sequence
  Chemically synthesized
tcagcgttcttcggaatg

SEQ ID NO: 537 - DNA, 18 bp, Artificial Sequence
  Chemically synthesized
gccttaccgcagagacag

SEQ ID NO: 538 - DNA, 18 bp, Artificial Sequence
  Chemically synthesized
tttcgcttttccgtttac

SEQ ID NO: 539 - DNA, 18 bp, Artificial Sequence
  Chemically synthesized
ttcgcttttccgtttacg

SEQ ID NO: 540 - DNA, 18 bp, Artificial Sequence
  Chemically synthesized
tcgcttttccgtttacga

SEQ ID NO: 541 - DNA, 18 bp, Artificial Sequence
  Chemically synthesized
cgcttttccgtttacgat

SEQ ID NO: 542 - DNA, 18 bp, Artificial Sequence
  Chemically synthesized
gcttttccgtttacgata

SEQ ID NO: 543 - DNA, 18 bp, Artificial Sequence
  Chemically synthesized
cttttccgtttacgatat

SEQ ID NO: 544 - DNA, 18 bp, Artificial Sequence
  Chemically synthesized
gaatgaattctcgtaact

SEQ ID NO: 545 - DNA, 18 bp, Artificial Sequence
  Chemically synthesized
aatgaattctcgtaacta

SEQ ID NO: 546 - DNA, 18 bp, Artificial Sequence
  Chemically synthesized
atgaattctcgtaactac

SEQ ID NO: 547 - DNA, 18 bp, Artificial Sequence
  Chemically synthesized
tgaattctcgtaactaca

SEQ ID NO: 548 - DNA, 18 bp, Artificial Sequence
  Chemically synthesized
gaattctcgtaactacat

SEQ ID NO: 549 - DNA, 18 bp, Artificial Sequence
  Chemically synthesized
aattctcgtaactacata

SEQ ID NO: 550 - DNA, 18 bp, Artificial Sequence
  Chemically synthesized
attctcgtaactacatag

SEQ ID NO: 551 - DNA, 18 bp, Artificial Sequence
  Chemically synthesized
ttctcgtaactacatagc

SEQ ID NO: 552 - DNA, 18 bp, Artificial Sequence
  Chemically synthesized
tctcgtaactacatagca

SEQ ID NO: 553 - DNA, 18 bp, Artificial Sequence
  Chemically synthesized
ctcgtaactacatagcac

SEQ ID NO: 554 - DNA, 18 bp, Artificial Sequence
  Chemically synthesized
gtacgatcgagtgtacag

SEQ ID NO: 555 - DNA, 18 bp, Artificial Sequence
  Chemically synthesized
tacgatcgagtgtacagt

SEQ ID NO: 556 - DNA, 18 bp, Artificial Sequence
  Chemically synthesized
acgatcgagtgtacagtg

SEQ ID NO: 557 - DNA, 18 bp, Artificial Sequence
  Chemically synthesized
cgatcgagtgtacagtga

SEQ ID NO: 558 - DNA, 18 bp, Artificial Sequence
  Chemically synthesized
gatcgagtgtacagtgaa

SEQ ID NO: 559 - DNA, 18 bp, Artificial Sequence
  Chemically synthesized
atcgagtgtacagtgaac

SEQ ID NO: 560 - DNA, 29903 bp, SARS-CoV-2
  Wuhan-Hu-1
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa